SEQUENCE LISTING (36 sequences)

SEQ ID NO: 1 - DNA, 23 bp, Artificial Sequence
  source: /note=“Description of Artificial Sequence Synthetic primer”
agctctgaatgtccaccaggagc

SEQ ID NO: 2 - DNA, 22 bp, Artificial Sequence
  source: /note=“Description of Artificial Sequence Synthetic primer”
accaatacagtttgccctcgga

SEQ ID NO: 3 - DNA, 25 bp, Artificial Sequence
  source: /note=“Description of Artificial Sequence Synthetic primer”
cagtgtgtgtgagagagagagatgg

SEQ ID NO: 4 - DNA, 20 bp, Artificial Sequence
  source: /note=“Description of Artificial Sequence Synthetic primer”
gaggagggccagagtaatgt

SEQ ID NO: 5 - DNA, 20 bp, Artificial Sequence
  source: /note=“Description of Artificial Sequence Synthetic primer”
gtggccaaccaacaacctgt

SEQ ID NO: 6 - DNA, 24 bp, Artificial Sequence
  source: /note=“Description of Artificial Sequence Synthetic primer”
aatgggtatgccatacgctgttac

SEQ ID NO: 7 - DNA, 22 bp, Artificial Sequence
  source: /note=“Description of Artificial Sequence Synthetic primer”
taagagcgacgaaagccaaaac

SEQ ID NO: 8 - DNA, 18 bp, Artificial Sequence
  source: /note=“Description of Artificial Sequence Synthetic oligonucleotide”
tgtggtggtgctcggttc

SEQ ID NO: 9 - DNA, 18 bp, Artificial Sequence
  source: /note=“Description of Artificial Sequence Synthetic oligonucleotide”
gaccaagacaggccaaat

SEQ ID NO: 10 - DNA, 19 bp, Artificial Sequence
  source: /note=“Description of Artificial Sequence Synthetic oligonucleotide”
ggattaaatctcaaggcaa

SEQ ID NO: 11 - DNA, 20 bp, Artificial Sequence
  source: /note=“Description of Artificial Sequence Synthetic oligonucleotide”
ggatctcagtctcccgaaag

SEQ ID NO: 12 - DNA, 252 bp, Artificial Sequence
  source: /note=“Description of Artificial Sequence Synthetic consensus sequence”
  modified_base (49)..(221): a, c, t, g, unknown or other
acgaacgatagaaacaatgcatagccagaaacgtgtggtggtgctcggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatgggaagaaagtacctttaagaaatgggt

SEQ ID NO: 13 - DNA, 79 bp, Artificial Sequence
  source: /note=“Description of Artificial Sequence Synthetic oligonucleotide”
acgaacgatagaaacaatgcatagccagaaacgtgtggtggtgctcggaatgggaagaaagtacctttaagaaatgggt

SEQ ID NO: 14 - DNA, 252 bp, Artificial Sequence
  source: /note=“Description of Artificial Sequence Synthetic polynucleotide”
acgaacgatagaaacaatgcatagccagaaacgtgtggtggtgctcggttccggagtgattgggttgagttccgctcttatactcgcacgaaaagggtactctgttcacattttggccagagatttgcccgaagatgtttcatctcaaacatttgcatctccttgggctggagctaactggacaccatttatgactttgaccgacggaccaagacaggccaaatgggaagaaagtacctttaagaaatgggt

SEQ ID NO: 15 - DNA, 96 bp, Artificial Sequence
  source: /note=“Description of Artificial Sequence Synthetic consensus sequence”
  modified_base (46)..(88): a, c, t, g, unknown or other
ccaccycatcagaggacaataatgggtgtggattaaatctcaaggnnnnnnnnnnnnnnnnnnnnnnnnnnnnnnnnnnnnnnnnnnnaagaggtg

SEQ ID NO: 16 - DNA, 53 bp, Artificial Sequence
  source: /note=“Description of Artificial Sequence Synthetic oligonucleotide”
ccaccccatcagaggacaataatgggtgtggattaaatctcaaggaagaggtg

SEQ ID NO: 17 - DNA, 96 bp, Solanum lycopersicum
ccacctcatcagaggacaataatgggtgtggattaaatctcaaggcaacggagctcaggctcggtctacctggatctcagtctcccgaaagaggtg

SEQ ID NO: 18 - DNA, 81 bp, Artificial Sequence
  source: /note=“Description of Artificial Sequence Synthetic consensus sequence”
  modified_base (57)..(57): a, c, t, g, unknown or other
caccatttacgaacgatagaaacaatgcatagccagaaacgtgtggtggtgctcggntccggagtgattgggttgagttcc

SEQ ID NO: 19 - DNA, 80 bp, Artificial Sequence
  source: /note=“Description of Artificial Sequence Synthetic oligonucleotide”
caccatttacgaacgatagaaacaatgcatagccagaaacgtgtggtggtgctcggtccggagtgattgggttgagttcc

SEQ ID NO: 20 - DNA, 81 bp, Artificial Sequence
  source: /note=“Description of Artificial Sequence Synthetic oligonucleotide”
caccatttacgaacgatagaaacaatgcatagccagaaacgtgtggtggtgctcggttccggagtgattgggttgagttcc

SEQ ID NO: 21 - DNA, 174 bp, Artificial Sequence
  source: /note=“Description of Artificial Sequence Synthetic consensus sequence”
  modified_base (104)..(110): a, c, t, g, unknown or other
gtggacagctgtaatatttccacctcatcagaggacaataatgggtgtggattaaatctcaaggcaacggagctcaggctcggtctacctggatctcagtctcnnnnnnnaggtgaggagacttgccctgtgakytcgacaaaggttgatgagaagctgctcttccccttgcac

SEQ ID NO: 22 - DNA, 167 bp, Artificial Sequence
  source: /note=“Description of Artificial Sequence Synthetic polynucleotide”
gtggacagctgtaatatttccacctcatcagaggacaataatgggtgtggattaaatctcaaggcaacggagctcaggctcggtctacctggatctcagtctcaggtgaggagacttgccctgtgatttcgacaaaggttgatgagaagctgctcttccccttgcac

SEQ ID NO: 23 - DNA, 174 bp, Solanum lycopersicum
gtggacagctgtaatatttccacctcatcagaggacaataatgggtgtggattaaatctcaaggcaacggagctcaggctcggtctacctggatctcagtctcccgaaagaggtgaggagacttgccctgtgagctcgacaaaggttgatgagaagctgctcttccccttgcac

SEQ ID NO: 24 - DNA, 101 bp, Solanum lycopersicum
tcatcagaggacaataatgggtgtggattaaatctcaaggcaacggagctcaggctcggtctacctggatctcagtctcccgaaagaggtgaggagacttg

SEQ ID NO: 25 - DNA, 101 bp, Solanum lycopersicum
caagtctcctcacctctttcgggagactgagatccaggtagaccgagcctgagctccgttgccttgagatttaatccacacccattattgtcctctgatga

SEQ ID NO: 26 - protein, 34 aa, Solanum lycopersicum
SSEDNNGCGLNLKATELRLGLPGSQSPERGEETC

SEQ ID NO: 27 - DNA, 58 bp, Solanum lycopersicum
tcatcagaggacaataatgggtgtggattaaatctcaaggaagaggtgaggagacttg

SEQ ID NO: 28 - DNA, 57 bp, Solanum lycopersicum
tcatcagaggacaataatgggtgtggattaaatctcaagaagaggtgaggagacttg

SEQ ID NO: 29 - DNA, 20673 bp, Artificial Sequence
  source: /note=“Description of Artificial Sequence Synthetic polynucleotide”
aaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggcctgttatccctaacaagtttgtacaaaaaagcaggctccgcggccgccttgtttaactttaagaaggagcccttcaccagctccgagtatagcccaacttcaccatatagctgtcaaaccttttataccactgccttggagactgcttaagtccatataaggacttcttcaacttgcagacgtgattttccttccctggaacttggaaaccatccggctgagtcatgtatatctcttcctccaactctccatgtagaaacgctgtcttcacatcaagttgttcaagctccagattctgatgtgcaactatcgctagtaacactcggatggaagtatgtctgaccactggtgagaagatctcattatagtccactccctctctttggttgaaacctctggcaacaaccctggctttatacttgactccttctgctggtgatatcccttccttcttcttgaaaacccatttgcaagtaataatctttctccccgaaggctgtatgaccagatcccatgtctgattcttgtgtagggactccatctcatctcccatagcggcaaaccatttttcagaatcagaacttaaaatggcttctttgtaagtagacggctcagatgtatctacctcttcagcaacctgcagtgcataacccaccatgtcctcaaaaccatacctcgtaggtggccgaactccaaccctccttggccgatcttgagctatactctgatggatatctgatggcatagattctggaatatcagtttcagtctgtggctcttgatcctcctcttcaggttcctttaaatcgctctcgttctgaatgacttgaaactccacctgtttgtcaagactcccagtttctgacgtagttgtaggcttcacaatggttctaagcagaggactttcatcaaagacaacgttcctgctcataataaccctcttttctgctggagaccagattctgaaacctttcactccatctccgtagcccacaaatactccctttttagctcttggttctaacttaccttcactgacgtgatagtaagccgtacaaccaaaagctttcagatttgaataatcagcagcttttccagaccacatctccataggtgtcttgcactgtatacctgtatgtggtccgcggttaatcaagtagcaagctgtactaaccgcttctgcccagaatcttctatctagcccagcattagagagcatgcaccttgctctctccagaagtgtttgattcatccgctcagctacaccgttctgctgtggtgtatttctgactgtgcgatgtcgagcaatcccttcatccttacagaattgatcaaattcagaccaacagaattccagcccattatcagttcgcaacctcttgatcttcttccctgtttgattttccatcaaaattttccactccttgaacttctggaaggcttcacttttatgcttcatcatgtacacccaagtcatccttgagtagtcatcaataatggacacaaaaaatctgcagcctcccaaagactcaacacggcatggaccccagcaatcagaatggatataatcaagtgtgccttttgttctatgaatggcctttggaaacttgttgcgatgtagttttccaaaaacacaatgttcacaaaactctaggctcttaaccttatgaccagcaagtaaatcctcctttgacagaatttgcatccctctttcacccatatgaccaagtcttatgtgccataacttagtcatatccttctggtgaaattctgacgatgcaacatgggctgaacctgtaaccgtggaaccttgtagaaaatacaaagtaccacgcatgacacctttcagaatcaaatttgaacccttccagacccgcaagactccatcttttcccgaccagctgaatcccttgctgtccaaaagactgagagatatcagatttttcgtcatcaatggaacgtgcctgacctcgttcaatgtgcagaagctaccgtcatgtgtccttatcttgatcgagcctgtcccaaccaccttgcagacagaactgttggccatcgagatgctgcctccgtctacctgctcataagtcgtgaaccactctctcctaggacagatgtgataggatgccccagaatcaagaacccacacatctgaatgatgagtgtgctcatccgcaactagggcaatatcttcttcagaattggtgtcttcttcagcaacagcagcagacactgattgtttttccgattgcttcttcttcttcggacaatcaaatttccaatgtcccttctccttgcagtaattacaaacatcatccggctttgcacccttcgacatcggcttatttttctttccgccgtttttccttccctttctgctactggtgaacagaccggaaggctgtatgtccgtacttgtgccgttagccttatgccgtaattccctgctatgaagggctgatctgacttcttccagtgacacagtatctttcccaacaatgaacgattgaacaaaattctcaaacgacattgggagagatactaacagaatcagggcagcatcttcatcctcgatcttcacatcgatattacgcaattctaataacaaagtattcaattgctctaagtgttccctgagttgtgtaccttcagccattcgtaaaccgaatagacgttgtttcagaagcagcttgttggttagagattttgtcatgtacaaactctccagcttcaaccacagaccagcagcagtctcttcatccgagacctccgtgatgacgtcatccgcgagacacagcatgatcgtcgagtgcgccttttcctccagaatcgccatctcaggagtaacgacggcgttcttgtctttcgacaacggcgcccagaagccttgctgtttcaacaaggcccgcatcttgatctgccataaactgaaactgttcctccctgtgaatttgtcgattttcacgttcaaagcagacatctcgaattctccaagaacaccgattaaccgagaggctctgataccaatttgttgtgcggaatttgagataatacgagaaaatataaacgcgaaaaataagacaacagatttacgtggttcaccaacaaattggctacgtccacgggaagagagggagcagttttattatggagaggcaaaaacagaattacagaatagggtttcccatagcgtctatatatagtgctaagctacgccctaacaggcttgggcccaacatacagaatcaacagaaaattaagggcccaatacaacaacattgtataccgtcggcccgggggcgtctccgcccccccggacccccaggccagggggcgcgtcgcccccctggaccccccgactcgctgaccgggcagcgagacccccgtcctttctgtttgtagcgggtccgattcaaggcattcaacacaagaaaaagacaataaagttataaatagttccctatcccaatagggtaaatctttcttcaaatatttttattctaaacaactcgttagatttttttttccttttcatttattcaagaattttaacctgaactttttttttaaaaaaaagaaaactattaaattttcccttaaaataattaaaatatccttaaaattattacttaaaaaattataaatactaaaaaacatatacacaaaagattacgacagaagatatatggaatggaagagatttagcatcacaatttggaaagagtattttgaaaattagaaaaaaaaaattgggataaaagtttctcgaagatgcaaataaaaagtcaagaaattaatctattccaaattgattaaaatatattttatcaaatttaaaatatttataaatgaaaatttatacattaaacatgaccaacttatgattttgtctgattttaatgttttttaggtgattagcaatctaagtaatttattttgaaagtgcttataaaattaagatggaaatttgaaaatatcagtttttgaagtttgaatggtgtgtgaaatatttttgtaggatttaaaacaatacataggataagtgaaatgaaatttcatccaaatttatgtaaattgtcaaaaattgattaaagtttatgaacaaaaaacaatttgtaatttctttaaaaattcttcctccaaaatctagagaaaatgtcattgacggtttcttaactattcgagtagggtttaaatagtttcttaactattcatttataggatttaatctttctaattgctcaaaatattaatatatttgatcttttaactatttacttaccgatcgattttggtcagacctaccatagagcttagtctcttaaagatgtgaatctcaagattttctactgctatatccactcacatcacaattcacacctttgttgtgcgtctatttttttcccctctccctctaaaaaagataagttttcttcacgttttattcttgaaaagtagtatttttgttaatttactactagtataatataatgtgtacgctaatctaatttttaatataaaataatgatataaagtttaatttagtggttgaaagaagaagcaaagcaaaaagaatcatattcaattaagggacaaataggtctcaagttataatattggtaaggaactctcactcttgtctgttttccaataacccaaaaaccaagaaacacataaaaacacagaagaaattacactgcacctcgatttgatcattctaatctaaaaataaaaattaaactgtatatctctctctcactaggagaggagggatattggcagataatgacagtgtttcagtggcagtgaagaaaaaaggggtatgagttttcattgggaaggtaaaaattttgcacccaaatacaagctaaccctttaacatgttcatgtttttcatagtctttgcttcttgctacaacactatagagagaaaaaaaaaagaaaagaaaagtgaacaatacactgttttttactaattattttttagaaaaagaaaaaaggaatattgtgtgtttgcttttttttctgactagtagtattgctaactatgtattccattaaggatttgctgtgaaaaagcctgatatcagtaagcataaaactcgggagatcacttacacacacacaccctcgtaaaaaagagaagagagatttactgttaaacagaggtttttttccatttcttttttttttctcagtgtgtgtgagagagagagatggttttcataggcaaaaacaaatagaaaggaacaaaatttagagtgaagaagaaagtgtgtgagagaataagggtgggcgcgcctctcaacacaacatatacaaaacaaacgaatctcaagcaatcaagcattctacttctattgcagcaatttaaatcatttcttttaaagcaaaagcaattttctgaaaattttcaccatttacgaacgatagaaacaatgcatagccagaaacgtgtggtggtgctcggttccggagtgattgggttgagttccgctcttatactcgcacgaaaagggtactctgttcacattttggccagagatttgcccgaagatgtttcatctcaaacatttgcatctccttgggctggagctaactggacaccatttatgactttgaccgacggaccaagacaggccaaatgggaagaaagtacctttaagaaatgggtagaacttgttcctaccggccatgccatgtggctcaaagggactagacgatttgcacaaaatgaagatggtctattgggtcattggtataaagacataacacctaattatagacctttgccaagctctgaatgtccaccaggagccatcggcgttacttacgatactttgtctgttcatgctccaaaatattgccaatatctggctcgtgaattgcagaagttgggagcaactttcgagaggcgaacagttacttcactagagcaagcttttgacggtgccgatcttgttgttaatgcaactggattaggcgctaaatcaatcgctggaatagacgaccaggcagccgaacctatccgagggcaaactgtattggtgaaaagtccatgcaagagatgtacaatggattcaagtgatcctgctagcccagcctacatcattcccaggccaggaggagaggtaatttgtggtggcacatacggagttggagactgggatctttcagttaatcccgaaaccgttcaaaggattctgaagcattgtctacgactagacccaactatatcatctgacggtactattgaaggaatagaggtccttaggcataatgtgggacttcgtcctgctcgaagaggaggacctagagttgaagccgagagaatagttcttcctctggaccgaacaaagtccccattgtcccttggacgtggatcagcaagagctgcaaaagagaaagaggttactttagttcatgcttacggtttttcatctgctggataccaacaatcttggggagcagcagaagacgttgcacagctcgtggacgaagcatttcagaggtatcacggagctgcaagggagagtaagctttgacggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctggcgcgccgacccagctttcttgtacaaagtggttcgataattccgatccagcctaggcccgggcctgaggacgcgtccatggttaattaagacgtcgaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatc

SEQ ID NO: 30 - DNA, 20697 bp, Artificial Sequence
  source: /note=“Description of Artificial Sequence Synthetic polynucleotide”
aaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggcctgttatccctaacaagtttgtacaaaaaagcaggctccgcggccgccttgtttaactttaagaaggagcccttcaccagctccgagtatagcccaacttcaccatatagctgtcaaaccttttataccactgccttggagactgcttaagtccatataaggacttcttcaacttgcagacgtgattttccttccctggaacttggaaaccatccggctgagtcatgtatatctcttcctccaactctccatgtagaaacgctgtcttcacatcaagttgttcaagctccagattctgatgtgcaactatcgctagtaacactcggatggaagtatgtctgaccactggtgagaagatctcattatagtccactccctctctttggttgaaacctctggcaacaaccctggctttatacttgactccttctgctggtgatatcccttccttcttcttgaaaacccatttgcaagtaataatctttctccccgaaggctgtatgaccagatcccatgtctgattcttgtgtagggactccatctcatctcccatagcggcaaaccatttttcagaatcagaacttaaaatggcttctttgtaagtagacggctcagatgtatctacctcttcagcaacctgcagtgcataacccaccatgtcctcaaaaccatacctcgtaggtggccgaactccaaccctccttggccgatcttgagctatactctgatggatatctgatggcatagattctggaatatcagtttcagtctgtggctcttgatcctcctcttcaggttcctttaaatcgctctcgttctgaatgacttgaaactccacctgtttgtcaagactcccagtttctgacgtagttgtaggcttcacaatggttctaagcagaggactttcatcaaagacaacgttcctgctcataataaccctcttttctgctggagaccagattctgaaacctttcactccatctccgtagcccacaaatactccctttttagctcttggttctaacttaccttcactgacgtgatagtaagccgtacaaccaaaagctttcagatttgaataatcagcagcttttccagaccacatctccataggtgtcttgcactgtatacctgtatgtggtccgcggttaatcaagtagcaagctgtactaaccgcttctgcccagaatcttctatctagcccagcattagagagcatgcaccttgctctctccagaagtgtttgattcatccgctcagctacaccgttctgctgtggtgtatttctgactgtgcgatgtcgagcaatcccttcatccttacagaattgatcaaattcagaccaacagaattccagcccattatcagttcgcaacctcttgatcttcttccctgtttgattttccatcaaaattttccactccttgaacttctggaaggcttcacttttatgcttcatcatgtacacccaagtcatccttgagtagtcatcaataatggacacaaaaaatctgcagcctcccaaagactcaacacggcatggaccccagcaatcagaatggatataatcaagtgtgccttttgttctatgaatggcctttggaaacttgttgcgatgtagttttccaaaaacacaatgttcacaaaactctaggctcttaaccttatgaccagcaagtaaatcctcctttgacagaatttgcatccctctttcacccatatgaccaagtcttatgtgccataacttagtcatatccttctggtgaaattctgacgatgcaacatgggctgaacctgtaaccgtggaaccttgtagaaaatacaaagtaccacgcatgacacctttcagaatcaaatttgaacccttccagacccgcaagactccatcttttcccgaccagctgaatcccttgctgtccaaaagactgagagatatcagatttttcgtcatcaatggaacgtgcctgacctcgttcaatgtgcagaagctaccgtcatgtgtccttatcttgatcgagcctgtcccaaccaccttgcagacagaactgttggccatcgagatgctgcctccgtctacctgctcataagtcgtgaaccactctctcctaggacagatgtgataggatgccccagaatcaagaacccacacatctgaatgatgagtgtgctcatccgcaactagggcaatatcttcttcagaattggtgtcttcttcagcaacagcagcagacactgattgtttttccgattgcttcttcttcttcggacaatcaaatttccaatgtcccttctccttgcagtaattacaaacatcatccggctttgcacccttcgacatcggcttatttttctttccgccgtttttccttccctttctgctactggtgaacagaccggaaggctgtatgtccgtacttgtgccgttagccttatgccgtaattccctgctatgaagggctgatctgacttcttccagtgacacagtatctttcccaacaatgaacgattgaacaaaattctcaaacgacattgggagagatactaacagaatcagggcagcatcttcatcctcgatcttcacatcgatattacgcaattctaataacaaagtattcaattgctctaagtgttccctgagttgtgtaccttcagccattcgtaaaccgaatagacgttgtttcagaagcagcttgttggttagagattttgtcatgtacaaactctccagcttcaaccacagaccagcagcagtctcttcatccgagacctccgtgatgacgtcatccgcgagacacagcatgatcgtcgagtgcgccttttcctccagaatcgccatctcaggagtaacgacggcgttcttgtctttcgacaacggcgcccagaagccttgctgtttcaacaaggcccgcatcttgatctgccataaactgaaactgttcctccctgtgaatttgtcgattttcacgttcaaagcagacatctcgaattctccaagaacaccgattaaccgagaggctctgataccaatttgttgtgcggaatttgagataatacgagaaaatataaacgcgaaaaataagacaacagatttacgtggttcaccaacaaattggctacgtccacgggaagagagggagcagttttattatggagaggcaaaaacagaattacagaatagggtttcccatagcgtctatatatagtgctaagctacgccctaacaggcttgggcccaacatacagaatcaacagaaaattaagggcccaatacaacaacattgtataccgtcggcccgggggcgtctccgcccccccggacccccaggccagggggcgcgtcgcccccctggaccccccgactcgctgaccgggcagcgagacccccgtcctttctgtttgtagcgggtccgattcaaggcattcaacacaagaaaaagacaataaagttataaatagttccctatcccaatagggtaaatctttcttcaaatatttttattctaaacaactcgttagatttttttttccttttcatttattcaagaattttaacctgaactttttttttaaaaaaaagaaaactattaaattttcccttaaaataattaaaatatccttaaaattattacttaaaaaattataaatactaaaaaacatatacacaaaagattacgacagaagatatatggaatggaagagatttagcatcacaatttggaaagagtattttgaaaattagaaaaaaaaaattgggataaaagtttctcgaagatgcaaataaaaagtcaagaaattaatctattccaaattgattaaaatatattttatcaaatttaaaatatttataaatgaaaatttatacattaaacatgaccaacttatgattttgtctgattttaatgttttttaggtgattagcaatctaagtaatttattttgaaagtgcttataaaattaagatggaaatttgaaaatatcagtttttgaagtttgaatggtgtgtgaaatatttttgtaggatttaaaacaatacataggataagtgaaatgaaatttcatccaaatttatgtaaattgtcaaaaattgattaaagtttatgaacaaaaaacaatttgtaatttctttaaaaattcttcctccaaaatctagagaaaatgtcattgacggtttcttaactattcgagtagggtttaaatagtttcttaactattcatttataggatttaatctttctaattgctcaaaatattaatatatttgatcttttaactatttacttaccgatcgattttggtcagacctaccatagagcttagtctcttaaagatgtgaatctcaagattttctactgctatatccactcacatcacaattcacacctttgttgtgcgtctatttttttcccctctccctctaaaaaagataagttttcttcacgttttattcttgaaaagtagtatttttgttaatttactactagtataatataatgtgtacgctaatctaatttttaatataaaataatgatataaagtttaatttagtggttgaaagaagaagcaaagcaaaaagaatcatattcaattaagggacaaataggtctcaagttataatattggtaaggaactctcactcttgtctgttttccaataacccaaaaaccaagaaacacataaaaacacagaagaaattacactgcacctcgatttgatcattctaatctaaaaataaaaattaaactgtatatctctctctcactaggagaggagggatattggcagataatgacagtgtttcagtggcagtgaagaaaaaaggggtatgagttttcattgggaaggtaaaaattttgcacccaaatacaagctaaccctttaacatgttcatgtttttcatagtctttgcttcttgctacaacactatagagagaaaaaaaaaagaaaagaaaagtgaacaatacactgttttttactaattattttttagaaaaagaaaaaaggaatattgtgtgtttgcttttttttctgactagtagtattgctaactatgtattccattaaggatttgctgtgaaaaagcctgatatcagtaagcataaaactcgggagatcacttacacacacacaccctcgtaaaaaagagaagagagatttactgttaaacagaggtttttttccatttcttttttttttctcagtgtgtgtgagagagagagatggttttcataggcaaaaacaaatagaaaggaacaaaatttagagtgaagaagaaagtgtgtgagagaataagggtgggcgcgcctctcaacacaacatatacaaaacaaacgaatctcaagcaatcaagcattctacttctattgcagcaatttaaatcatttcttttaaagcaaaagcaattttctgaaaattttcaccatttacgaacgatagaaacaatggcttcatacccctgtcatcaacatgcttcagctttcgatcaagcagctcgttcccgagggcataataatcgtcgtacagctttacgtccaaggcgtcagcaaaaagcaactgaagtaaggctagaacaaaaaatgccaactttactccgagtttacatagatggtcctcacggtatgggtaaaactactacaacccagctcctggtggccttgggaagccgtgatgacattgtttatgtgcctgaacctatgacttactggcgtgtcctaggtgcttcagagactatcgcaaatatctatactactcaacatagattagatcagggggagattagtgcaggagatgcagccgttgtgatgacatccgcacaaattacaatgggtatgccatacgctgttacagatgctgttcttgcacctcacattggaggtgaagctggctcatcacacgcacctcctccagcacttactctgatctttgacaggcatccaatagctgcacttctctgttatccagcagcaagatatttgatgggatcaatgactccacaagcagttttggctttcgtcgctcttattcctccaacattacccggaacaaacatagtccttggtgcactacctgaagataggcacattgatagattggcaaagcgtcagcgacctggggagaggttagatctggccatgttggcagcaatcagacgtgtttatggtctcctagccaacacagtaaggtatctacaaggcggtggttcatggcgtgaagactgggggcagttaagtggtgctgccgtcccccctcaaggggctgaaccacaatcaaacgcaggcccaaggccccacattggtgacaccctttttaccctgtttagagcaccagaacttctagcccctaatggcgacctttataatgtttttgcctgggctttggatgttttggctaagagacttagaccaatgcatgttttcatacttgattacgatcaatctccagctggttgcagagatgctttgttgcaacttacttctggcatggtgcagacacatgtcacaacaccaggatctatcccaacaatatgtgatctcgctagaacatttgctcgtgagatgggggaagctaactaacggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacatggcgcgccgacccagctttcttgtacaaagtggttcgataattccgatccagcctaggcccgggcctgaggacgcgtccatggttaattaagacgtcgaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatc

SEQ ID NO: 31 - DNA, 11960 bp, Artificial Sequence
  source: /note=“Description of Artificial Sequence Synthetic polynucleotide”
caagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccctaggacaacagcaacatctggagcttttcgtctagttgtttaggaatttattctcttgttcttaaattgtaaacaactcctaaatctataaaggaattggtatgttgtgttaaaagtctaggttgtccaggtgggataccttagtgggtagattgtttttctactttggcttgttgagcaatagaggtctattgcttaacggtaagattgataactctttcttacgtttggtgtaatcgtgtttcgcttttgcttttaaagattagtgaaaacgattgaaaatcctgtgggacaggtcgtggttttactcccttaagcaaggagtttttcacgtaaaatcattgtgttgattttactgcatttaactttctgataattttctgaagtaaagtaagggacctggtccattactaataagtgaagccatacattctatcaattatataatgtgtatatacatatattatactttaacgcttgctaacaagctcttcttaggtccaaaatgccccataagttgcattggaatcatttgggataaacaatattaaaaggtaattattatgtgaaagatgctccttcctttgggcttaatttgttatttgtgatcacatgatggaataactaactaactaattgatattgaaattaattttacttagtgggaatataatatttgcaatagcaggcggccgcggagcctgcttttttgtacaaacttgtgatatcactagtgcggccgcctgcaggtcgataatacgactcactatagggagaccggcagatctgccgtcgatttggaaatctccccattcgattgtatctccgtccttgtcgatgtacgatttgacgtcggagctcgatttagctccctgaatattcggatggaaatgtgctgaccgggttggggagaccaggtcgaagaatctgttattcgtgcactggtactttccttcgaactgaacaagcacatggagatgaggttccccattttcatgaagctctctgcaaattttgatgaatttcttattgactggggtatttaggttttgtaattgggaaagtgcttcttctttagacaaagagcactgtggataagtgaggaaatagttctttgactgaactctaaatttctttggtgggggcatttttgtaataagaaggggtactccagatgagttactccaattgagccttctcaaacttgctcattcaattggagtattagagtaacttatatataagaaccctctatagaactattaatctggttcatacacgtggcggccatccgatataatattaccggatggccgcgcgcttttttttaatccgtacagtccaatactctcacatccaatcataatgcgtcgtacaagcctatatatttccaacaacttgggccttaagttgttggaggcccattataaattaaagtgatcttggcccaatgtctttaactcaaagtcgacactagtggatccgatatcctgcagccatgactagtgatatcacaagtttgtacaaaaaagcaggctctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgaacaaagcaccagtggtctagtggtagaatagtaccctgccacggtacagacccgggttcgattcccggctggtgcaggtgtggtggtgctcggttcgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggatctcagtctcccgaaaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagggaccaagacaggccaaatgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagggattaaatctcaaggcaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagttttagagctagaaatagcaagttaaaataaggctagtccgttatcaacttgaaaaagtggcaccgagtcggtgctttttttctagacccagctttcttgtacaaagttggcattaacccagctttcttgtacaaagtggtcatggatgcattctagagaattcgatcacgaattaataaaatttgaattttattgaatgattctccaatacatgatttacatacactctgtttgttgcgaaacgtacagctctaattacattgtttattgaaataacacctaattgatctagatacatgttgactaaatatctaaatctagctaaataaattgatccagaagctgtcatccacgtcgtccaaacttggaagttcaggtaggctttgtggagatgcaacgctttcctcaggttgtggttgaaccgtatctgtacgtggtataccctcgttctggtatacagtggttcctctaccctgtgtatcttgaaataaaggggattttttagctcccagatatacacgccattctccgcctgatgtgcagtgatgagttcccctgtgcgtgaatccatgtcccgcacagtctaagtggaagtagatggagcacccgcactgtagatcaaccctgcgccttctgattgcccgtttcttggcttgcctgtgtgctgtcttgatagaggggggctgtgagggtgatgaagatcgcattcttgacagtccagttctttagacctgtattttctgctttgtctaagaactctttatagctggcaccctcaccaggattgcaaagcacgattgctgggattccgcctttaatttgaactggcttaccgtacttgcaatttgattgccaatctttctgggcccctagcaattctttccagtgctttagctttagataatgcggtgcgatgtcatcaatgacgttatactgcacatcattcgagaagactcgaccattgaagtctaggtgtccactgagatagttatgtgggcctaacgcacgtgcccacatcgtcttccctgttcttgaatcaccctcgacgatgatacttacaggtctctctggccgcgcagctgcacccgtcccgaaataattatccgcccattcctgcatctcgtcaggaacgttagtgaaagaagagacttgaaatggaggaacccacggttccggagcctttgcgaatattcgttctaggttagagcggatgttatgattttgtaatacaaaatcttttggttgttcttcccttagaactgctaaggcagattgaaccgaacttgcatttaacgctttcgcatatgaatcattagcagactgctggcctcctctggcagatctgccgtcgatctggaaatctccccattcgattgtatctccgtccttgtcgatgtacgatttgacgtcggagctcgatttagctccctgaatattcggatggaaatgtgctgaccgggttggggagaccaggtcgaagaatctgttattcgtgcactggtactttccttcgaactgaacaagcacatggagatgaggttccccattttcatgaagctctctgcaaattttgatgaatttcttattgactggggtatttaggttttgtaattgggaaagtgcttcttctttagacaaagagcactgtggataagtgaggaaatagttctttgactgaactctaaatttctttggtgggggcatttttgtaataagaaggggtactccagatgagttactccaattgagccttctcaaacttgctcattcaattggagtattagagtaacttatatataagaaccctctatagaactattaatctggttcatacacgtggcggccatccgatataatattaccggatggccgcgcgcttttttttaatccgtacagtccaatactctcacatccaatcataatgcgtcgtacaagcctatatatttccaacaacttgggccttaagttgttggaggcccattataaattaaagtgatcttggcccaagcttcagctgctcgagttctatagtgtcacctaaatcgtatgtgtatgtcgaccatatgggagagct

SEQ ID NO: 32 - DNA, 11476 bp, Artificial Sequence
  source: /note=“Description of Artificial Sequence Synthetic polynucleotide”
ataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttacctcagggtacgagaaactctttattcacagagaactttatatcttaacagatttaattgagagagtgagtaagttctttaacttagctcaggatgtggtagaagcaagttttgagtatgccaaggttgaagagaggttaggtcacgtcagaaacgtgttgcaactggcgggtggaaaatccacgaatgccgatttgacaattaagatttctgacgatgtcgaacaactgcttggaaaacgtggtggattcttgaaggttgtgaacggtatcttgagcaagaatggtagtgacgtagtcactaacgacaatgagcttattcatgcaattaaccaaaatctggtaccagataaagtcatgtctgtgtcgaacgtaatgaaagagactgggtttctgcagtttccaaagtttttatctaagttggaaggacaggtaccgaaaggaacaaaatttctagacaaacacgttcctgattttacttggatacaagctcttgaagaaagagtgaatattcggagaggagaatcgggacttcagactctattagctgatatcgttccgaggaatgctattgctgctcagaaattgacaatgctaggttacatcgagtatcacgactatgtggtgatcgtctgtcagtctggagtatttagtgacgattgggcgacatgtagaatgctttgggcagcactatctagtgctcaactatatacctatgttgacgccagtagaatcggtccaatcgtttacggttggttattgtgattggttgatgaggaaattctgtttgaaggctggttgaaagtaccagggcgggagaagccatattctctatcgttgtaggaagcgattgaaataattcctgtggtcacgtcgcacgagcatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacgagctcgagcaccgaagacctattgaacaaagcaccagtggtctagtggtagaatagtaccctgccacggtacagacccgggttcgattcccggctggtgcaggtgtggtggtgctcggttcgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggatctcagtctcccgaaaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagggaccaagacaggccaaatgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagggattaaatctcaaggcaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagttttggtcttcatggatccaatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcctaggccaccatgttgggcccggcgcgccaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 33 - DNA, 12745 bp, Artificial Sequence
  source: /note=“Description of Artificial Sequence Synthetic polynucleotide”
caagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccctaggacaacagcaacatctggagcttttcgtctagttgtttaggaatttattctcttgttcttaaattgtaaacaactcctaaatctataaaggaattggtatgttgtgttaaaagtctaggttgtccaggtgggataccttagtgggtagattgtttttctactttggcttgttgagcaatagaggtctattgcttaacggtaagattgataactctttcttacgtttggtgtaatcgtgtttcgcttttgcttttaaagattagtgaaaacgattgaaaatcctgtgggacaggtcgtggttttactcccttaagcaaggagtttttcacgtaaaatcattgtgttgattttactgcatttaactttctgataattttctgaagtaaagtaagggacctggtccattactaataagtgaagccatacattctatcaattatataatgtgtatatacatatattatactttaacgcttgctaacaagctcttcttaggtccaaaatgccccataagttgcattggaatcatttgggataaacaatattaaaaggtaattattatgtgaaagatgctccttcctttgggcttaatttgttatttgtgatcacatgatggaataactaactaactaattgatattgaaattaattttacttagtgggaatataatatttgcaatagcaggcggccgcggagcctgcttttttgtacaaacttgtgatatcactagtgcggccgcctgcaggtcgataatacgactcactatagggagaccggcagatctgccgtcgatttggaaatctccccattcgattgtatctccgtccttgtcgatgtacgatttgacgtcggagctcgatttagctccctgaatattcggatggaaatgtgctgaccgggttggggagaccaggtcgaagaatctgttattcgtgcactggtactttccttcgaactgaacaagcacatggagatgaggttccccattttcatgaagctctctgcaaattttgatgaatttcttattgactggggtatttaggttttgtaattgggaaagtgcttcttctttagacaaagagcactgtggataagtgaggaaatagttctttgactgaactctaaatttctttggtgggggcatttttgtaataagaaggggtactccagatgagttactccaattgagccttctcaaacttgctcattcaattggagtattagagtaacttatatataagaaccctctatagaactattaatctggttcatacacgtggcggccatccgatataatattaccggatggccgcgcgcttttttttaatccgtacagtccaatactctcacatccaatcataatgcgtcgtacaagcctatatatttccaacaacttgggccttaagttgttggaggcccattataaattaaagtgatcttggcccaatgtctttaactcaaagtcgacactagtggatccgatatcctgcagccatgactagtgatatcacaagtttgtacaaaaaagcaggctccgcggccgcccccttcaccatggatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtctgtacccgatcaacaccgagacccgtggcgtcttcgacctcaatggcgtctggaacttcaagctggactacgggaaaggactggaagagaagtggtacgaaagcaagctgaccgacactattagtatggccgtcccaagcagttacaatgacattggcgtgaccaaggaaatccgcaaccatatcggatatgtctggtacgaacgtgagttcacggtgccggcctatctgaaggatcagcgtatcgtgctccgcttcggctctgcaactcacaaagcaattgtctatgtcaatggtgagctggtcgtggagcacaagggcggattcctgccattcgaagcggaaatcaacaactcgctgcgtgatggcatgaatcgcgtcaccgtcgccgtggacaacatcctcgacgatagcaccctcccggtggggctgtacagcgagcgccacgaagagggcctcggaaaagtcattcgtaacaagccgaacttcgacttcttcaactatgcaggcctgcaccgtccggtgaaaatctacacgaccccgtttacgtacgtcgaggacatctcggttgtgaccgacttcaatggcccaaccgggactgtgacctatacggtggactttcaaggcaaagccgagaccgtgaaagtgtcggtcgtggatgaggaaggcaaagtggtcgcaagcaccgagggcctgagcggtaacgtggagattccgaatgtcatcctctgggaaccactgaacacgtatctctaccagatcaaagtggaactggtgaacgacggactgaccatcgatgtctatgaagagccgttcggcgtgcggaccgtggaagtcaacgacggcaagttcctcatcaacaacaaaccgttctacttcaagggctttggcaaacatgaggacactcctatcaacggccgtggctttaacgaagcgagcaatgtgatggatttcaatatcctcaaatggatcggcgccaacagcttccggaccgcacactatccgtactctgaagagttgatgcgtcttgcggatcgcgagggtctggtcgtgatcgacgagactccggcagttggcgtgcacctcaacttcatggccaccacgggactcggcgaaggcagcgagcgcgtcagtacctgggagaagattcggacgtttgagcaccatcaagacgttctccgtgaactggtgtctcgtgacaagaaccatccaagcgtcgtgatgtggagcatcgccaacgaggcggcgactgaggaagagggcgcgtacgagtacttcaagccgttggtggagctgaccaaggaactcgacccacagaagcgtccggtcacgatcgtgctgtttgtgatggctaccccggagacggacaaagtcgccgaactgattgacgtcatcgcgctcaatcgctataacggatggtacttcgatggcggtgatctcgaagcggccaaagtccatctccgccaggaatttcacgcgtggaacaagcgttgcccaggaaagccgatcatgatcactgagtacggcgcagacaccgttgcgggctttcacgacattgatccagtgatgttcaccgaggaatatcaagtcgagtactaccaggcgaaccacgtcgtgttcgatgagtttgagaacttcgtgggtgagcaagcgtggaacttcgcggacttcgcgacctctcagggcgtgatgcgcgtccaaggaaacaagaagggcgtgttcactcgtgaccgcaagccgaagctcgccgcgcacgtctttcgcgagcgctggaccaacattccagatttcggctacaagaactgaaagggtgggcgcgccgacccagctttcttgtacaaagtggtcatggatgcattctagagaattcgatcacgaattaataaaatttgaattttattgaatgattctccaatacatgatttacatacactctgtttgttgcgaaacgtacagctctaattacattgtttattgaaataacacctaattgatctagatacatgttgactaaatatctaaatctagctaaataaattgatccagaagctgtcatccacgtcgtccaaacttggaagttcaggtaggctttgtggagatgcaacgctttcctcaggttgtggttgaaccgtatctgtacgtggtataccctcgttctggtatacagtggttcctctaccctgtgtatcttgaaataaaggggattttttagctcccagatatacacgccattctccgcctgatgtgcagtgatgagttcccctgtgcgtgaatccatgtcccgcacagtctaagtggaagtagatggagcacccgcactgtagatcaaccctgcgccttctgattgcccgtttcttggcttgcctgtgtgctgtcttgatagaggggggctgtgagggtgatgaagatcgcattcttgacagtccagttctttagacctgtattttctgctttgtctaagaactctttatagctggcaccctcaccaggattgcaaagcacgattgctgggattccgcctttaatttgaactggcttaccgtacttgcaatttgattgccaatctttctgggcccctagcaattctttccagtgctttagctttagataatgcggtgcgatgtcatcaatgacgttatactgcacatcattcgagaagactcgaccattgaagtctaggtgtccactgagatagttatgtgggcctaacgcacgtgcccacatcgtcttccctgttcttgaatcaccctcgacgatgatacttacaggtctctctggccgcgcagctgcacccgtcccgaaataattatccgcccattcctgcatctcgtcaggaacgttagtgaaagaagagacttgaaatggaggaacccacggttccggagcctttgcgaatattcgttctaggttagagcggatgttatgattttgtaatacaaaatcttttggttgttcttcccttagaactgctaaggcagattgaaccgaacttgcatttaacgctttcgcatatgaatcattagcagactgctggcctcctctggcagatctgccgtcgatctggaaatctccccattcgattgtatctccgtccttgtcgatgtacgatttgacgtcggagctcgatttagctccctgaatattcggatggaaatgtgctgaccgggttggggagaccaggtcgaagaatctgttattcgtgcactggtactttccttcgaactgaacaagcacatggagatgaggttccccattttcatgaagctctctgcaaattttgatgaatttcttattgactggggtatttaggttttgtaattgggaaagtgcttcttctttagacaaagagcactgtggataagtgaggaaatagttctttgactgaactctaaatttctttggtgggggcatttttgtaataagaaggggtactccagatgagttactccaattgagccttctcaaacttgctcattcaattggagtattagagtaacttatatataagaaccctctatagaactattaatctggttcatacacgtggcggccatccgatataatattaccggatggccgcgcgcttttttttaatccgtacagtccaatactctcacatccaatcataatgcgtcgtacaagcctatatatttccaacaacttgggccttaagttgttggaggcccattataaattaaagtgatcttggcccaagcttcagctgctcgagttctatagtgtcacctaaatcgtatgtgtatgtcgaccatatgggagagct

SEQ ID NO: 34 - DNA, 11596 bp, Artificial Sequence
  source: /note=“Description of Artificial Sequence Synthetic polynucleotide”
ataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttacctcagggtacgagaaactctttattcacagagaactttatatcttaacagatttaattgagagagtgagtaagttctttaacttagctcaggatgtggtagaagcaagttttgagtatgccaaggttgaagagaggttaggtcacgtcagaaacgtgttgcaactggcgggtggaaaatccacgaatgccgatttgacaattaagatttctgacgatgtcgaacaactgcttggaaaacgtggtggattcttgaaggttgtgaacggtatcttgagcaagaatggtagtgacgtagtcactaacgacaatgagcttattcatgcaattaaccaaaatctggtaccagataaagtcatgtctgtgtcgaacgtaatgaaagagactgggtttctgcagtttccaaagtttttatctaagttggaaggacaggtaccgaaaggaacaaaatttctagacaaacacgttcctgattttacttggatacaagctcttgaagaaagagtgaatattcggagaggagaatcgggacttcagactctattagctgatatcgttccgaggaatgctattgctgctcagaaattgacaatgctaggttacatcgagtatcacgactatgtggtgatcgtctgtcagtctggagtatttagtgacgattgggcgacatgtagaatgctttgggcagcactatctagtgctcaactatatacctatgttgacgccagtagaatcggtccaatcgtttacggttggttattgtgattggttgatgaggaaattctgtttgaaggctggttgaaagtaccagggcgggagaagccatattctctatcgttgtaggaagcgattgaaataattcctgtggtcacgtcgcacgagcatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacatgaggaagaggacgctcgtgtccgttttgttccttttctcgcttctgtttcttcttccagatcaaggtacgtttcgatgtatatatgtgttgattttgagtagatagatataagtgttgcttaggattcatatgctgatgatggcgatatttctgtggtgtttatcataggaagaaagttacacgcgaatgcagaagacgtcgtcgacatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccctgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatcatgatgagctataaatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcctaggccaccatgttgggcccggcgcgccaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 35 - DNA, 3689 bp, Solanum lycopersicum
atgtctccgccgctccttggtgttggggaggaggagggccagagtaatgtaactctactggcttcttcaacttccttaggaagcatatgcataaaaggatcagctcttaaagagcgaaactatatgggtctatctgattgttcgtcggtggacagctgtaatatttccacctcatcagaggacaataatgggtgtggattaaatctcaaggcaacggagctcaggctcggtctacctggatctcagtctcccgaaagaggtgaggagacttgccctgtgagctcgacaaaggttgatgagaagctgctcttccccttgcacccttccaaagatactgctttctcggtatcgcagaaaacagtagttagtggcaacaaacgaggattttcagacgctatggatggattctcagaggtgaactatcatttctttaataaggttactcattttttggtaaagctgcaattgaagttacataatctatcctacaggggaagtttctgtcgaattccggtgtgaaagcaggtgatacaaaggagacctcacgtgtgcaaccacctaaaatgaaagatgctaatactcagagtacagttccagagaggccttctgctgtgaatgatgcctcaaaccgtgcgggcagtggtgcccctgctacaaagtgagtatctccccactaggagtaagaagtgtgatatttggataatgattgtacaaaatgtctaatctgtatcttggattaattttagggcacaggttgttggttggccacccattcgatcttttagaaagaacactctagcctctgcctcgaagaataacgaagaggttgacggaaaagctggctcaccagctctttttattaaggtcagcatggatggtgctccctatttgaggaaagtggacctcagaacctgttctgcataccaggagctatcttctgctcttgaaaaaatgttcagctgttttacaataggttagctattttggagcctttagtagctcataatttcattgattggattaaaatagtaagatacatgttcatgatttttccattgccctagttactgctacttccttgttatgacaggtcaatatggatctcatggagctcctgggaaggatatgttaagtgagagcaaattgaaggatttgcttcatggatctgagtatgtcctcacttacgaagataaggatggggactggatgcttgtcggtgatgtcccctgggagtaagctcttgctaattcaactcctttttcttttcttcccgtttagcactgtttggtatagtttactacattttcggccaacctatttcttggttgtatgctttttccattgagataaaaagaagaatatagtgcacgtggtgtggatttagtagtaaatgtggacttggtgcaactgatagtaaatacaactcatcttcagattcagaaataaaattagactagtcagtcctcatcttaaggaagttaaacaagttcttgtataactgcaagcttagagagcaatgatgctctagtaagtcctaaattaaagtggaaatctcaaacctgacccataattaggttctctgaattgagaagccttgccatgcagtaagagttcaggtaggtttggccttgtgttacaaggaagatcttttttttttgattgatagagcttttaaggagattgattataatgaagtctgattgaattaataaatcagcaagttaattttgagggccatcattactacttaccattttagaaaactcatggctcattttgtatgtgtataatgctatgtatgaaaatatttgccatggttaagcatgtagtcaggatttttatcaatcaagattcatgctgccttcagttatggattataatttcttatctctagcatgaatagaaaaaccaataataatctgattttttgataatgccaaaggattaagggatgataaacgtaactagaagaatttggtatcattgttggtttgacactcaactaggattcaatgatattgctagtgtgtgttagatagcgattgcgctgctttgggaaaaaatgcctaataatccgtgtgacattcaactcttgcttcacatgtgtcccattgttagtggtgctattccatgtaatttgcacagcttgattcagggagttagtcctctttctgtctctctctagttcgctttatctcccttcttttttgttcttttgcaatgatgaatctggtggtctactataacaatggtagacctgaacatattgttttcttgaatctgttctctcttggaccatataaaaataccagtagtacatttattaagggctgttctttgcaagttctaatatcttaggttcagacttgtaataaattttattgaatgtagtttctgtttgattgtttgtatatttgacgatgaccatgcttttaattcctaatattttcatgttgtaggatgtttatcgatacttgcaaaaggttgaggatcatgaaaggttcagatgccattggcctgggtgagtatctcttgttgagccttagacctcttcattcacacaatcaaaatgaaaaacggaaagaaagaccgtctcctccttcatctcccccgaccttgttcttagggcttgtacttctgtttttgtattttgacatgcataaccatttggcgagagatgtggggcttgctcaagtggttcagcacctccaccatcaactggtaggttgaggtttagagttcgagtcatgctggagtgttagtgggaatactagtgatcttccgggagggtagaaaaaaaggggaaaaacaactacttggtgataaatagaaatatacaaactgtaacgcagaattatctggtttcaacatttgatttctgtgaatcttgattactttctatgatttgaaacaaaacttccttgcaagtttcatatttaaaagatattattgaagaaataattagaagtcatgaaatgctggtttaggaattctcccacccagctgttcgtcatgaaagttctctgaattaccgtgatgcattttttcaagtagcgttaagtattagagaattgcttgcctgacagaactacatttgttatggatctggcctcattttattatttgtcaagcggcaatagaaagatttaggacatcaaggtaggctaggcatcttgaaatggtggtggtaggttcatttgcattgtaggacacacctgcatctgtgagagatgaggatggagagtgaaggagaatggttaatgtagtcccaaaaccgaaaggaaacgagctataagttattgtaaatctatgggcagtgttatgcagttattgctgttttaaacatgtgagttagtcagctttatctcattataaaagggcaaaggggataacaaactcagctgttataatttccgcaaaagttctgttacccaataatgcatttccatatgctgatttgtgtgcagacttcggataggtcttttattaatctgtaagtgtgtttagtctatggtaatgcaactctagtttaatttttggtattggttgcaacatttcaacgaccaactcctgctcctaaggtatgaatgattacctacgaaacggaaacatgttacaattccactaacttctgtgcatttttatgtgcagccccaagggctatggaaaagtgtcggagcagaaattag

SEQ ID NO: 36 - DNA, 2071 bp, Solanum lycopersicum
atgggaagagctccttgttgtgataagaataatgttaaaagagggccatggtcaccagaagaagatgctaagcttaaagaattcattgaaaaatatggaactggtggtaattggattgctcttcctctaaaagctggtaagtttagtcgaactcagtaacttatagttcaaaccttttgtatttattttagaaaattcacttaatatatgtacatccaataatataacaacaacaatatgtatagtgtaatttcatgaataagttaaacgaggataggatgttatatgcagatgttactcctacaaatgttgagtagaaaagtataaacccttagattcaaaagaaaggaagaattttgaaattagacaataaaatatcaggttacaaagcaaatgaagcaataaacaatagtaataaacactaaagaaaaagaaataacaaagagtacgttcgaaatttttagaaccaataaagttcaaatcttgaccataaaaagtatttcttttatattttctatttatataatttgatgagattatggttttaaatcaggattaaagagatgtggaaagagctgcagattaagatggctaaattatctaaggccaaatataaagcatggtgatttttctgatgaagaagatagggtaatatgcagtttatatgccagcattgggagcaggtgacaaactttttaattaagtcaaaattatttaacagtttttcctctcttttttttttcctgttttggtcactaattatctaccactaccttcattttaccttcaatttcttcactcatcatgtatttgcatatgacttttgagacaaaagtaaatcccataaattttaaaaaaagaaattgaaagaagaacttcaatggtgtagaaaataaatattaattaagactcaaagaaaagtaataatttaattttgttgggttctgactgaagattctacaatttttatggatatttcatctgcatgcatgccagttttagatcttggatcaagtccttaattacagctgttaggacacagtccataaaatgtaaaactattctaagtttcttgataaaacaacttatgtatagcacctacggttagatttctctaaaacaatatttattattacagtcaagataatttgaaatcaattttccatgtttataacaatactttgctaaaacaactaaaatatatccaaacaaacaattttattataaaaatatttaactatatatcttatcaatttgagtttaaattttctatgacatgtaacttttttgtaacaagtttgatatacttaaaagttgattaaattgaataataaacatctatagatggttaaatcaaattttaaaatgccaaattatttatatgttgaatatatattataacgtaaatgatgttgtacatttatacaggtggtcaattatagcagctcagttaccagggaggaccgacaatgatattaaaaactattggaacacaaagcttaagaagaagctcatgggttttattcagtcatcatctaatattaaccagagaactaaatcacctaatttattattccctcctacaagtactcttcaaacaacttttcaatcccaatctcaagcatcaatttcaaatcttttaagagattcatatgtagagcccattccactagtccaaccaaatttcatgtacaacaacaataacatgatgaactttcaattaggtacaaataatcaacattcttataattttcatgatcaaagcttaatgaatcccatgcaaacaattagttcttgttcttcatctgatggtctaagttgcaaacaaattagctatggcaatgaggaaatgatgtgtcaaattccttttgaagaaacccaaaagtttacacttgacaattattgtactacttgggctgatcatcaaaagacaaatggatattttgggaataactttcaaagtagtcaattccagtatgatgatcatactaatattgaagaaattaaggagttgattagtagtagctctagtaatggcaatggatgtaacaatgtagggtactggggttaa

(19) United States
(12) Patent Application Publication
(10) Pub. No.: US 2021/0348177 A1
(43) Pub. Date: Nov. 11, 2021

(54) GENERATION OF HERITABLY GENE-EDITED PLANTS WITHOUT TISSUE CULTURE

(71) Applicant: The Regents of the University of California, Oakland, CA (US)

(72) Inventors: Mily RON, Davis, CA (US); Neelima R. SINHA, Davis, CA (US); Anne B. BRITT, Davis, CA (US); Moran FARHI, Davis, CA (US)

(21) Appl. No.: 17/272,874

(22) PCT Filed: Sep. 5, 2019
(86) PCT No.: PCT/US2019/049670

§ 371 Date: Mar. 2, 2021

Related U.S. Application Data
(60) Provisional application No. 62/727,431, filed on Sep. 5, 2018.

(51) Int. Cl. C12N 15/82; C12N 9/22; C12N 15/86; C12N 15/11

(52) U.S. Cl. CPC C12N 15/8203; C12N 9/22; C12N 15/86; C12N 2310/20; C12N 15/8213; C12N 15/8207; C12N 2770/00043; C12N 15/111

(57) ABSTRACT

Methods and compositions for selecting plants with targeted nuclease alterations are provided.

### CROSS-REFERENCE TO RELATED PATENT APPLICATIONS

The present patent application claims priority to U.S. Provisional Patent Application No. 62/727,431, filed Sep. 5, 2018, which is incorporated by reference.

### STATEMENT AS TO RIGHTS TO INVENTIONS MADE UNDER FEDERALLY SPONSORED RESEARCH AND DEVELOPMENT

This invention was made with government support under contract no. NSF EAGER 1636397 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 27, 2019, is named 081906-1153085-230410PC_SL.txt and is 133,928 bytes in size.

### BACKGROUND OF THE INVENTION

Plant breeding relies on selection from natural or induced genetic variation, which is a limiting factor. The vastly increasing genetic knowledge enables accelerated improvements of crops. New biotechnological tools enable identification, cloning, and modification of specific genetic loci that influence desired traits such as mass yield, plant architecture, biotic/abiotic stress resistance and nutritional values.

One of the most exciting new developments in this regard is sequence-specific genome engineering or genome editing. This approach is based on the delivery of molecular scissors to the plant nucleus in order to mutagenize specific locus/loci in the genome. These DNA double-strand breaks (DSBs) can be sites of mutation via error-prone host repair pathways or can serve as sites of DNA integration by homologous or nonhomologous recombination. Until 2012 two methods for genome editing were available i.e. zinc finger nucleases (ZFNs) and TAL effector nucleases (TALENs). However, neither was widely adopted by the research community, as they require complex and time-consuming engineering and assembly of each nuclease. Similarly, neither method was amenable to multiplexing, i.e. simultaneous editing of several different targets, and therefore these were not suitable for high-throughput applications. Shortly after the discovery of bacterial CRISPR (clustered regularly interspaced short palindromic repeats)/Cas (CRISPR-associated sequence) type II prokaryotic adaptive immune system, it was demonstrated that CRISPR-cas9 can be used as an efficient method for genome engineering in eukaryotes, including plants.

In the type II CRISPR/Cas9 system of Streptococcus pyogenes, Cas9 encodes a DNA nuclease that acts in a sequence specific manner after forming a complex with CRISPR RNA (crRNA) and trans-activating crRNA (tracrRNA) noncoding RNAs. crRNA/tracrRNA activated Cas9 is guided to sequences matching the crRNA and preceding protospacer adjacent motif (PAM) where it induces a break in the DNA. The application of CRISPR/Cas9 to genome engineering is facilitated by combining the crRNA, tracrRNA with a single, easily determined guide RNA (sgRNA), which defines a very specific, often unique, target site in the genome.

CRISPR in plants: CRISPRs have been used to delete, add, activate, and suppress targeted genes in many organisms, demonstrating the broad applicability of this technology (Ma et al., 2015; Raitskin & Patron, 2016). We previously used CRISPR/Cas9 to demonstrate that SHR function is evolutionarily conserved between Arabidopsis and tomato with respect to regulation of its downstream targets (SCR) and root length (Ron et al., 2014). More recently, Brooks et al. showed that CRISPR/Cas9 is highly efficient at generating targeted mutations in tomato; homozygous deletions of a desired size can be created in the first generation, and there is high efficiency of multiplex mutants generated by a single sgRNA that targets 2 genes simultaneously. These studies demonstrate that the CRISPR/Cas9 system provides a facile means to test gene function in plant development and is very efficient in tomato, one of our target crops (Brooks, Nekrasov, Lippman, & Van Eck, 2014).

Shortcomings of current approaches: Tomato transformations can be routinely done but are time consuming, taking approximately 6 months until T0 plants can be moved to soil and almost a year before T1 plants are ready for analysis. The transformation protocols have been standardized but are laborious and require personnel with extensive training and experience. Other crops in the Solanaceae (except tobacco) are much harder to transform and transformations are usually performed in a small number of specialized facilities. Soybean has been notoriously recalcitrant and tools for precise genome editing in this crop have lagged behind. This is also the case for other crops in the Fabaceae (pea, bean, chickpea).

Current approaches for genome editing rely on transgenic experiments for each gene to be targeted for modification. This makes the protocol expensive in terms of time and personnel costs, and inaccessible to small research labs. Some important crops are highly heterozygous, like the polyploid species potato. These have to be propagated vegetatively to preserve the composition of desirable traits. Stable transformation of such species via tissue culture would require crosses to segregate out the Cas9 and lead to a reshuffling in traits. Targeting desired varieties transiently and regenerating edited plants that are otherwise identical to the parental variety would be a boon to such crops. Added to all these difficulties in transformation is the lack of public acceptance for transgenic crops and the regulatory scenario surrounding such crops. A recent decision from the USDA to not regulate plants that could otherwise have been developed through traditional breeding techniques, as long as they are not plant pests or developed using plant pests is particularly noteworthy (Perdue, 2018).

### Definitions

An “endogenous” or “native” gene or protein sequence, as used with reference to an organism, refers to a gene or protein sequence that is naturally occurring in the genome of the organism.

A “gene of interest” refers to any genomic or episomal DNA sequence in a cell that one desired to target for cleavage and possible alteration. In some embodiments, the gene can encode a protein. In some embodiments, the gene encodes a non-coding RNA. In some embodiments, the portion of the gene targeted is a promoter, enhancer, or coding or non-coding sequence.

A “RNA-guided nuclease” refers to a nuclease, which in combination with a sgRNA, targets a DNA sequence for cleavage. Generally, absent the sgRNA, the nuclease is inactive and does not cleave the DNA at the targeted site. Examples of such nucleases include for example Cas9 and other nucleases as discussed in the context of CRISPR herein.

A polynucleotide or polypeptide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid that encodes an RNA, which in turn may be non-coding (like a gRNA) or directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The terms “identical” or percent “identity,” in the context of two or more nucleic acids or polypeptide sequences, refer to two or more sequences or subsequences that are the same or have a specified percentage of nucleotides or amino acid residues that are the same, when compared and aligned for maximum correspondence over a comparison window, as measured using one of the following sequence comparison algorithms or by manual alignment and visual inspection. Two nucleic acid sequences or polypeptides are said to be “identical” if the sequence of nucleotides or amino acid residues, respectively, in the two sequences is the same when aligned for maximum correspondence as described below. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

An “expression cassette” refers to a nucleic acid construct that, when introduced into a host cell, results in transcription and/or translation of an RNA or polypeptide, respectively.

### BRIEF SUMMARY OF THE INVENTION

In some embodiments, a method of generating a plant comprising a mutation in a gene of interest is provided. In some embodiments, the method comprises,

providing a plant expressing a guided nuclease targeted to a gene of interest in the plant;
generating a wound at a location on the plant at which the guided nuclease is expressed;
allowing shoots to form from callus at the wound; and
selecting at least one shoot from the wound comprising a guided nuclease-induced mutation in the gene of interest.

In some embodiments, the guided nuclease is a sgRNA-guided nuclease and the plant expresses one or more sgRNA that guides the nuclease to the gene of interest. In some embodiments, the guided nuclease and the sgRNA are expressed transiently. In some embodiments, RNA encoding the guided nuclease and the sgRNA are expressed from the same transient vector. In some embodiments, the sgRNA, and optionally the RNA encoding the guided nuclease, is expressed from a transient vector. In some embodiments, the transient vector is a viral vector. In some embodiments, the viral vector is a tobacco Rattle Virus (TRV) vector or a Potato Virus X (PVX) vector.

In some embodiments, the providing comprises delivering the guided nuclease and the sgRNA to the plant. In some embodiments, the guided nuclease and the sgRNA are part of a ribonucleoprotein complex.

In some embodiments, the guided nuclease is expressed from an expression cassette integrated in the genome of the plant. In some embodiments, the guided nuclease is a sgRNA-guided nuclease and the plant transiently expresses one or more sgRNA that guides the nuclease to the gene of interest.

In some embodiments, the plant further expresses a template nucleic acid molecule that acts as a template for homology-directed recombination (HDR) at the gene of interest after the guided nuclease cleaves the gene of interest.

In some embodiments, the method further comprises before the generating, expressing a counter-selectable marker in the plant, wherein the counter-selectable marker is shoot meristem-specific, expressing at least one additional sgRNA at said location, wherein the at least one additional sgRNA targets a gene encoding the counter-selectable marker such that the RNA-guided nuclease inactivates the counter-selectable marker; and before the selecting, applying counter selection to the plant such that shoots generated at the wound that do not contain the at least one additional sgRNA have inhibited growth compared to shoots that contain the at least one addition sgRNA. In some embodiments, the counter-selectable marker is a protein that generates a toxic product to plant cell in which the counter-selectable marker is expressed when provided with a substrate. In some embodiments, the counter-selectable marker is D-amino acid oxidase and the substrate is a D-amino acid. In some embodiments, the counter-selectable marker is Herpes Simplex Virus-1 Thymidine Kinase (HSVtk) and the substrate is ganciclovir.

In some embodiments, the plant is a monocot. In some embodiments, the plant is a dicot.

In some embodiments, the RNA-guided nuclease is a Cas9 or Cpf1 polypeptide.

In some embodiments, the method further comprises regenerating a plant from a shoot selected as comprising the guided nuclease-induced mutation in the gene of interest.

In some embodiments, wherein the plant is knocked-out for, has reduced or inhibited expression of, has reduced or inhibited activity of, or contains an inactivating mutation in at least one of more of ku70, ku80, DNA ligase IV, polQ, or XRCC4 protein.

Also provided is a plant comprising callus at a wound site generated by removal of a shoot, the wound comprising guided nuclease targeting a gene of interest, wherein the callus comprises one or more shoot comprising a mutated copy of the gene of interest, wherein the mutated copy was generated by cleavage of the gene of interest by the guided nuclease. Thus, the mutated copy of the gene of interest would not be present but for the guided nuclease Accordingly, in some embodiments, some or all of the remaining portion of the plant (e.g., the roots) do not have a mutated copy of the gene of interest. In some embodiments, the guided nuclease is a sgRNA-guided nuclease and the plant expresses one or more sgRNA that guides the nuclease to the gene of interest. In some embodiments, the guided nuclease and the sgRNA are expressed transiently. In some embodiments, RNA encoding the guided nuclease and the sgRNA are expressed from the same transient vector. In some embodiments, the sgRNA, and optionally the RNA encoding the guided nuclease, is expressed from a transient vector. In some embodiments, the transient vector is a viral vector In some embodiments, the viral vector is a tobacco Rattle Virus (TRV) vector or a Potato Virus X (PVX) vector In some embodiments, the guided nuclease is expressed from an expression cassette integrated in the genome of the plant. In some embodiments, the guided nuclease is a sgRNA-guided nuclease and the plant transiently expresses one or more sgRNA that guides the nuclease to the gene of interest.

In some embodiments, the plant further expresses a template nucleic acid molecule that acts as a template for homology-directed recombination (HDR) at the gene of interest after the guided nuclease cleaves the gene of interest. In some embodiments, the plant further expresses in a shoot meristem-specific manner a counter-selectable marker, and the plant further expresses at least one additional sgRNA at said callus, wherein the at least one additional sgRNA targets a gene encoding the counter-selectable marker such that the RNA-guided nuclease inactivates the counter-selectable marker.

In some embodiments, the counter-selectable marker is a protein that generates a toxic product to plant cell in which the counter-selectable marker is expressed when provided with a substrate. In some embodiments, the counter-selectable marker is D-amino acid oxidase and the substrate is a D-amino acid. In some embodiments, the counter-selectable marker is Herpes Simplex Virus-1 Thymidine Kinase (HSVtk) and the substrate is ganciclovir.

In some embodiments, the plant is a monocot. In some embodiments, the plant is a dicot.

In some embodiments, the guided nuclease is a Cas9 or Cpf1 polypeptide.

In some embodiments, wherein the plant is knocked-out for, has reduced or inhibited expression of, has reduced or inhibited activity of, or contains an inactivating mutation in at least one of more of ku70, ku80, DNA ligase IV, polQ, or XRCC4 protein.

### BRIEF DESCRIPTION OF THE DRAWINGS

FIG. 1A-B. Tomato shoot regeneration upon decapitation. (A) Callus formed on the wound. The purple dots are the initiating shoots or leaves. (B) Regenerated shoots and leaves from the decapitated plant.

FIG. 2A-C. Designed processes of initiating and selecting for CRISPR mutagenesis. (A) A tomato seedling expressing the CSM as well as Cas9. (B) Delivering gRNAs to the plant by agrobacteria injection. (C) Applying selecting agents to the decapitated plant.

FIG. 3. Leaf shape of entire mutant. (A) Leaf shape of a wildtype tomato, which is compound, with many leaflets. (B) Leaf shape of an entire mutant (the right half of the leaf), which has reduced leaf complexity with fewer leaflets.

FIG. 4. Leaf shape of Potato Leaf (c) mutant. (A) Leaf shape of a wildtype tomato, which is compound, with many leaflets. (B) Leaf shape of a potato leaf mutant, which has reduced leaf complexity with fewer leaflets and less serrated.

FIG. 5A-C. Constructs of DAAO-Cas9 and HSVtk-Cas9. The structure of the T-DNA in CAS9-CSM vectors from the left border (LB) to the right border (RB) (A) The construct of pDe/Kan-Cas9-DAAO. Cas9 driven by the parsley ubiquitin promoter, with the pea 3A terminator; DAAO driven by LeT6 promoter, with the TEV enhancer, a plant like Kozak sequence and the 35S terminator; neomycin/kanamycin resistance gene (NPTII) driven by the nopaline synthase (NOS) promoter, with the NOS terminator. (B) The construct of pDe/Kan-Cas9-HSVtk. Cas9 driven by the parsley ubiquitin promoter, with the pea 3A terminator; HSVtk driven by LeT6 promoter, with the TEV enhancer, a plant like Kozak sequence and the 35S terminator; neomycin/kanamycin resistance gene (NPTII) driven by the nopaline synthase (NOS) promoter, with the NOS terminator (C) The construct of pMR317/Cas9-HSVtk. Neomycin/kanamycin resistance gene (NPTII) driven by the nopaline synthase (NOS) promoter, with the NOS terminator, the same Cas9 driven by the parsley ubiquitin promoter, with the AtHSP18.2 terminator; HSVtk driven by LeT6 promoter, with the TEV enhancer, a plant like Kozak sequence and the 35S terminator.

FIG. 6A-D. Structures of T-DNAs in pMP6, pMP4, pMR420 and pMR417. The structure of the T-DNA in gRNA vectors from the left border (LB) to the right border (RB): (A) The structure of the T-DNA in pMP6: Tomato Mottle Virus (ToMoV) common region; AtU6-26 promoter driving tRNA-gRNA structure of two spacers for HSVTK and two spacers for C; ToMoV AC3 (REN), AC2 (TrAP), AC1 (REP), ToMoV common region. (B) The structure of the T-DNA in pMP4: CaMV 35S promoter from pCASS2; TRV strain Ppk20 RNA2 5′-sequence; 2b gene; CP-sgP-PEBV, an enhancer region (PEBV, the promoter of Pea early browning virus); tRNA-gRNA structure, two spacers for DAAO and two spacers for ENTIRE; TRV strain Ppk20 RNA2 3′-sequence; NOS terminator. (C) The structure of the T-DNA in pMR420: ToMoV common region; AtU6-26 promoter driving tRNA-gRNA structure, two spacers for HSVTK and two spacers for C; ToMoV AC3 (REN), AC2 (TrAP), AC1 (REP), ToMoV common region. (D) The structure of the T-DNA in pMR417: it has the neomycin/kanamycin resistance gene (NPTII) driven by the nopaline synthase (NOS) promoter, with the NOS terminator, Cas9 driven by the parsley ubiquitin promoter, with the AtHSP18.2 terminator, ToMoV common region; AtU6-26 p driving tRNA-gRNA structure, two spacers for HSVTK and two spacers for C; ToMoV AC3 (REN), AC2 (TrAP), AC1 (REP), ToMoV common region.

FIG. 7A-B. Expression of pTAV-GUS and pTRV2e-RFP in tomato stems. (A) Expression of pTAV-GUS (pMR316_pTAVbinary_GUSPlus) in the target tomato stem, with GUS stained in green-blue. Left: control. Middle: T-DNA-encoded GUS. Right: GUS encoded on pTAV and delivered by agrobacteria injection. (B) Expression of pTRV2e-RFP (pTRV2e-ER_tagRFP) in the target tomato stem. Upper: under white light conditions. Lower: the same view of the same stem taken under the green filter for red fluorescence. All photos were taken using Zeiss Discovery V12 fluorescent stereoscope.

FIG. 8. Candidate regenerated shoots for mutations in ENTIRE.

FIG. 9A-D. Alignment of Sanger sequencing reads to the original sequences: mutations detected in DAAO and in ENTIRE. (A) 173 bp deletion in DAAO between the two gRNA targets (SEQ ID NOS 12-14, respectively, in order of appearance). (B) 43 bp deletion in ENTIRE between the two gRNA targets (SEQ ID NOS 15-17, respectively, in order of appearance). (C) 1 bp deletion in DAAO in one of the gRNA targets (DAAOspacer82) (SEQ ID NOS 18-20, respectively, in order of appearance). (D) 7 bp deletion in ENTIRE in one of the gRNA targets (ENTspacer1) (SEQ ID NOS 21-23, respectively, in order of appearance).

FIG. 10—CRISPR at the ENTIRE locus of T1 plants. Top: T1 plants show entire phenotype, WT leaf is on the right. Bottom: Sanger sequencing of the entire locus in Tl showed the mutation was inheritable (SEQ ID NOS 24-26, 24, 27, 28, and 28, respectively, in order of appearance).

FIG. 11—CRISPR at the POTATO LEAF (C) locus. Plants show c phenotype, WT plant is on the left.

### DETAILED DESCRIPTION OF THE INVENTION

### Introduction

The inventors have discovered a method of conveniently introducing guided nuclease-mediated genetic modifications in plants. The method does not require tissue culture and can be performed if desired with only transient expression procedures. Any plant-based system that induces generation of shoots or other plant parts can be used. A plant, either transiently or stably expressing a guided nuclease (e.g., if the nuclease is a CRISPR nuclease such as for example Cas9 or Cpf1, the guided nuclease is complexed with an sgRNA) is wounded at a location at which the guided nuclease is expressed, leading to the formation of callus comprising cells whose progenitors were exposed to the editing machinery. For example, after removing an apical bud (or otherwise wounding the plant to trigger shoots formation from the wound) from a plant, a wound will form that will develop into callus that will ultimately be the source of a number of new shoots. By expressing an RNA-guided (or protein-guided) nuclease and one or more sgRNA (or other guide molecules) in the wound site either prior to wounding or after, (e.g., in callus) the inventors have found that the plant will produce at least some shoots that contain genetic alterations induced by the guided nuclease, as targeted by at least one or more sgRNA or other guide molecule. Thus, shoots can be selected and propagated to generate plants having a desired genetic alteration. If desired, the efficiency of the method can be improved by inclusion of a selection method, for example a counter selection as described herein.

TABLE 1
Crop species that regenerate shoots in-vivo after
decapitation (based on Amutha et al., 2009)

| | | Regeneration | |
|---|---|---|---|
| | | efficiency | Recalcitrant to |
| Species | Common name | (%)^{a} | transformation^{b} |
| Brassica napus | rape, canola | 100 | − |
| Cucumis melo | cantaloupe, | 50 | + |
| | muskmelon | | |
| Cucurbita pepo | squash, pumpkin | 96 | + |
| Daucus carota | carrot | 21 | + |
| Gossypium hirsutum | cotton | 46 | − |
| Glycine max | soybean | 90 | + |
| Helianthus annuus | sunflower | 18 | + |
| Linum usitatissimum | flax | 96 | − |
| Papaver somniferum | poppy | 87 | + |
| Phaseolus vulgaris | common bean | 37 | + |
| Solanum lycopersicum | tomato | 97 | − |
| Spinacia oleracea | spinach | 60 | − |
| Vigna unguiculata | cowpea | 52 | − |

This work provides two-pronged benefits—the first is to make CRISPR mediated gene editing accessible to many research labs working on crops in many plant families and the second is to develop non-transgenic CRISPR technology for these crops. A major challenge in genome editing is selecting cells and cell lines that are mutated, to obviate screening large numbers of transgenic plants. To date, no effective selection method has been deployed. In order to generate gene-edited plants without tissue culture, we utilize the ability of many plants to regenerate shoots upon decapitation (Amutha et al., 2009—Table 1, above), coupled with transient expression of the CRISPR/Cas9 system using disarmed viral replicons. Many crops are susceptible to viral infections. We have targeted viral replicons that infect species within many families to make the system more generally applicable to plants in these families.

In some embodiments, a variant of the methods described herein can be employed that is designed to improve the frequency of mutagenesis at the target. In this variant, starting transgenic stock that can be used by the research community is deployed, allowing one to easily identify and isolate tissues that have experienced high levels of CRISPR induced mutagenesis. Studies reported that genomic editing by CRISPR/Cas9 in one genomic site coincided with changes in another when several gRNAs are used simultaneously (Cermak et al., 2017; Liao, Tammaro, & Yan, 2015). We have deployed a counter-selectable marker (CSM) to facilitate identification of occurrences of successful Cas9 activity. For example, to select for tomato plants with edited genomes, we have developed tomato lines that express a conditionally lethal gene that encodes an enzyme that transforms a harmless chemical into a toxic one, functioning as a counter selectable marker (CSM). CRISPR-mediated targeted co-mutagenesis at the marker and a gene of interest and selection resulted in development of shoots resistant to the chemical. Application of the selection compound kills tissues that have not been edited by Cas9 and enables the generation of genome-edited plants without the need for tissue culture. This CSM system can be deployed in many plant species, once the appropriate transgenic CSM line has been generated.

### Nucleases

One goal of the methods described herein is for the guided nuclease and any nuclease-guiding nucleic acid to be expressed at the wound site, for example in cells that are progenitors of callus generated from the wound such that new shoots from the callus will include a targeted mutation in a gene of interest caused by the guided nuclease.

A “guided nuclease” refers to a DNA nuclease that is targeted to a particular genomic DNA sequence, for example by a separate small guide RNA (sgRNA) or a fused protein sequence that targets the DNA sequence. Any method of delivery can be used to deliver the nuclease and guide molecule if separate from the nuclease. In some embodiments, the nuclease and a guide RNA are delivered by the same mechanism. In some embodiments, the nuclease is delivered to the plant by one mechanism and the sgRNA is delivered to the plant by a second mechanism.

Any nuclease that can be targeted to a particular genome sequence to induce sequence-specific cleavage and thus allow for targeted mutagenesis can be used. Exemplary nucleases include, for example, TALE nucleases (TALENs), zinc-finger proteins (ZFPs), zinc-finger nucleases (ZFNs), DNA-guided polypeptides such as Natronobacterium gregoryi Argonaute (NgAgo), and CRISPR/Cas RNA-guided polypeptides including but not limited to Cas9, CasX, CasY, Cpf1, Cms1, MAD7 and the like.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known. For example, the amino acid sequence of S. pyogenes Cas9 protein may be found in the SwissProt database under accession number Q99ZW2. In some embodiments, the CRISPR enzyme has DNA cleavage activity, such as Cas9. In some embodiments the CRISPR enzyme is Cas9, and may be Cas9 from S. Pyogenes, S. aureus or S. pneumonia or Actinobacteria, Aquificae, Bacteroidetes-Chlorobi, Chlamydiae-Verrucomicrobia, Cvanobacteria, Firmicutes, Proteobacteria, Spirochaetes, or Thermotogae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. Non-limiting examples of mutations in a Cas9 protein are known in the art (see e.g., WO2015/161276), any of which can be included in a CRISPR/Cas9 system in accord with the provided methods. Cpf1 use in higher plants is described in, e.g., Begemann, M B, et al., Sci Rep. 2017; 7: 11606. CMS1 is described in, for example, Begemann, M B, et al., Characterization and Validation of a Novel Group of Type V, Class 2 Nucleases for in vivo Genome Editing, BioRxiv (2018)(doi.org/10.1101/192799).

Plant gene manipulations can be precisely tailored in non-transgenic organisms using the CRlSPR/Cas9 genome editing method. In this bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA. Binding of these RNAs to Cas9 involves specific sequences and secondary structures in the RNA. The two RNA components can be simplified into a single element, the single guide-RNA (sgRNA), which is transcribed from a cassette containing a target sequence defined by the user. In this system the nuclease creates DNA breaks at the target region programmed by the sgRNA. These can be repaired by non-homologous recombination, which often yields inactivating mutations. The breaks can also be repaired by homologous recombination, which enables the system to be used for gene targeted gene replacement. Accordingly, in one aspect, a method can be provided using CRISPR/Cas9 or Cpf1 or Cms1 or other nuclease as described above to introduce at least one of the mutation into a plant cell using the methods described herein.

### Guide Molecules

Separately, in the case of CRISPR-based nucleases, a guide nucleic acid (e.g., one or more sgRNA) that guides the nuclease to a target genome sequence can be expressed in the plant at the wound site, for example in the progenitor cells that give rise to callus cells leading to the formation of the shoot meristem or axillary meristems, such that shoots later emerging from the callus will arise from cells having active nuclease and guide molecules expressed therein.

The guide nucleic acid can target any genome sequence in the cell as desired. In some embodiments, more than one guide molecule will be expressed to target more than one different genomic target sequences. Guide RNA sequence selection can be performed as previous described. See, e.g., PCT Publication No. WO2018107028.

In some embodiments, the target sequence in the gene of interest may be complementary to the guide region of the sgRNA. In some embodiments, the degree of complementarity or identity between a guide region of a sgRNA and its corresponding target sequence in the gene of interest may be about 50%, 55%, 60%, 65%, 70%, 75%, 80%, 85%, 90%, 95%, 96%, 97%, 98%, 99%, or 100%, with higher or 100% identity being most desirable to avoid off-target effects. In some embodiments, the guide region of a sgRNA and the target region of a gene of interest may be 100% complementary or identical. In other embodiments, the guide region of a sgRNA and the target region of a gene of interest may contain at least one mismatch. For example, the guide region of a sgRNA and the target sequence of a gene of interest may contain 1, 2, 3, 4, 5, 6, 7, 8, 9, or 10 mismatches, where the total length of the target sequence is at least about 17, 18, 19, 20 or more base pairs. In some embodiments, the guide region of a sgRNA and the target region of a gene of interest may contain 1-6 mismatches where the guide sequence comprises at least about 17, 18, 19, 20 or more nucleotides. In some embodiments, the guide region of a sgRNA and the target region of a gene of interest may contain 1, 2, 3, 4, 5, or 6 mismatches where the guide sequence comprises about 20 nucleotides. The 5′ terminus may comprise nucleotides that are not considered guide regions (i.e., do not function to direct a Cas9 or another nuclease protein to a target nucleic acid (e.g., gene of interest).

Alternatives to CRISPR-based nucleases also can be used. Exemplary nucleases guided by a protein or DNA molecule include, for example, TALE nucleases (TALENs), zinc-finger proteins (ZFPs), zinc-finger nucleases (ZFNs), each of which can be covalently or non-covalently linked to a nuclease), and DNA-guided polypeptides such as Natronobacterium gregoryi Argonaute (NgAgo). Examples of ZFNs, TALEs, and TALENs are described in, e.g., Lloyd et al., Frontiers in Immunology, 4(221), 1-7 (2013).

In some embodiments, the DNA-targeting molecule comprises one or more zinc-finger proteins (ZFPs) or domains thereof that bind to DNA in a sequence-specific manner and that are fused to a nuclease. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

In some embodiments, the DNA-targeting molecule is or comprises a zinc-finger DNA binding domain, TALEN, or other DNA-targeting protein fused to a DNA cleavage domain to form a targeted nuclease. In some embodiments, fusion proteins comprise the cleavage domain (or cleavage half-domain) from at least one Type IIS restriction enzyme and one or more DNA-targeting protein. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J Biol. Chem. 269:31,978-31,982.

Introduction of the nuclease and in the case of CRISPR-based methods or other methods requiring a separate guide molecule, introduction of the nuclease and separate guide molecule can be achieved in any number of ways as desired. In some embodiments, the nuclease, the guide molecule, or both are introduced in the plant via a transient method that does not result in introduction of coding sequences for the nuclease or guide nucleic acids into the plant genome. In some embodiments, the nuclease and guide molecule are introduced by the same mechanism. For example, a CRISPR nuclease and a sgRNA can be introduced into the plant in the form of a ribonucleoprotein complex (see, e.g or encoded by DNA or RNA introduced into the plant, wherein the nuclease and optionally the sgRNA are expressed from the introduced DNA or RNA. Alternatively, in some embodiments, an expression cassette encoding the nuclease can be introduced into the genome of the plant and a separate guide molecule, if needed by the nuclease used, can be introduced transiently. A number of methods for introducing nucleases and guide molecules are described in for example, Cermak, T., et al., The Plant Cell, Vol. 29: 1196-1217 (June 2017)

In some embodiments, the nuclease and optionally the guide molecule, can be expressed from a constitutive or substantially ubiquitous promoter. For example, a promoter or promoter fragment can be employed to direct expression of the nuclease in all or substantially all (e.g., many tissues and including shoot meristem) tissues of a plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, the parsley UBI promoter (Kawalleck et al., Plant Mol Biol. (1993 February) 21(4):673-84), RPS5 (Hiroki Tsutsui et al. Plant and Cell Physiology (2016)); 2X35SΩ (Belhaj, Khaoula, et al. Plant methods 9.1 (2013): 39); AtUBI10 (Callis J, et al. Genetics 139: 921-939 (1995)); SlUBI10 (Dahan-Meir, Tal, et al. The Plant Journal (2018)); G10-90 (Ishige, Fumiharu, et al. The Plant Journal 18.4 (1999): 443-448) and other transcription initiation regions from various plant genes known to those of skill.

The guide molecule can be expressed from an expression cassette that has been introduced into a plant cell such that the expression cassette is present in the progenitor cells that will make callus or new shoots at the wound.

The resulting DNA breakpoint can be repaired by the cell's DNA repair mechanism (e.g., via non-homologous end joining), which will frequently introduce one or more insertion or deletion at the breakpoint, thereby harming or eliminating activity of encoded proteins or RNAs. In some embodiments, a nucleic acid template molecule can be introduced into the cell (on the same or a separate vector as the guide RNA) such that the nucleic acid template molecule is used by the cell as a homologous template for DNA repair via homology-directed repair (HDR). If the nucleic acid template molecule is homologous but contains one or more nucleotide changes from the cell's chromosomal DNA, the repair will introduce those nucleotide changes as part of the repair, thereby introducing specific targeted changes to the target DNA.

An expression cassette for expression of the nuclease, the guide molecule, or both can be part of a viral replicon or non-viral vector that is introduced into the plant. Any vector with or without a viral replicon can be used. Exemplary plant viral replicon vectors include parts from, e.g., DNA viruses (Bean yellow dwarf virus, Wheat dwarf virus, Cabbage leaf curl virus, and Potato Virus X (PVX)) and RNA viruses (Tobacco rattle virus). See, e.g., Zaidi et al., Front Plant Sci. 2017; 8: 539 (2017) and Lacomme et al., Curr Protoc Microbiol. 2008 February; Chapter 16:Unit 161.

Any method of delivery of the guide molecules to the plant is contemplated. For example, instead of the use of viral replicon vectors, one can directly deliver nuclease and RNA complexes as RiboNucleoProteins (RNPs). In another embodiment, one can use particle gun bombardment at the wound site, or in the progenitor cells that will make callus or incipient meristems, to introduce the guide molecule, the nuclease, or both, or nucleic acids encoding the nuclease and/or guide molecule directly to the plant.

Alternatively, a DNA construct may be combined with suitable T-DNA flanking regions and introduced into a conventional Agrobacterium tumefaciens host vector. The virulence functions of the Agrobacterium tumefaciens host will direct the transfer of the T-DNA into plant cells when the cell is infected by the bacteria. Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Microinjection techniques can also be used. These techniques are well known in the art and thoroughly described in the literature. The introduction of DNA constructs using polyethylene glycol precipitation is described for example in Paszkowski et al. EMBO J. 3:2717-2722 (1984). Electroporation techniques are described for example in Fromm et al. Proc. Natl. Acad. Sci. USA 82:5824 (1985). Ballistic transformation techniques are described for example in Klein et al. Nature 327:70-73 (1987). In some embodiments, silicon carbide whisker-mediated plant transformation is employed (see, e.g., Asad and Arshad (2011). Silicon Carbide Whisker-mediated Plant Transformation, Properties and Applications of Silicon Carbide, Prof. Rosario Gerhardt (Ed.), ISBN: 978-953-307-201-2).

### Wound Generation

As noted above, the methods involve in some embodiments, in generating a wound in the plant that will later generate a plurality of shoots. Generation of the wound can be achieved as desired. In some embodiments, the wound is in an aerial portion of the plant, e.g., in a shoot. In some embodiments, the shoot that is removed comprises the apical bud, thereby “decapitating” the plant. Shoot decapitation in the stem or hypocotyl or epicotyl, or runners, or internodes, seedlings or woody buds will generate a wound and reprogramming to produce axillary buds or callus and new shoots. The location of the wound should be at a location in which the nuclease and any guiding molecules are expressed. Thus in some embodiments, the wound is formed at the location at which the nuclease and/or guide molecule(s) have been introduced to the plant.

### Screening for Shoots Comprising Desired Genetic Modification

Following introduction of the wound, new meristem will form to produce new shoots at the wound site. At least some cells in the wound region will contain both the nuclease and the targeting molecule such that the nuclease cleaves chromosomal DNA in the cells at the target DNA sequence. The resulting shoots will contain the desired genomic mutation at the gene of interest. Screening for shoots that include the cleavage event can be performed for example visually (for example if the change results in a visual phenotype) or by molecular genetic testing (e.g., PCR-based or other sequence-based detection of DNA from a shoot). Notably, the methods can be performed in the absence of tissue culture or formation of protoplasts.

Once the shoots have been identified, they can be transferred to soil or rooting media and allowed to root and produce seed, which will include the desired introduced alteration at the target nucleic acid. Alternatively, one can propagate the shoot by cuttings or other vegetative and clonal propagation methods.

### Counter Selection

In some embodiments, a counter selection strategy can be used to enrich for shoots that include the guide molecule and the nuclease. For example, an expression cassette comprising a shoot meristem-specific promoter operably linked to a counter-selectable marker can be introduced into the target plant. The expression cassette is introduced before the wounding of the plant. The counter-selectable marker will generate a sensitivity of the plant to an external agent that can be introduced at a desired time. In addition, at least one additional sgRNA or other guide molecule can be introduced with the guide molecule (e.g., sgRNA) for the target nucleic acid (e.g., gene of interest), wherein the at least one additional sgRNA targets a gene encoding the counter-selectable marker such that the guided nuclease inactivates the counter-selectable marker when introduced into a cell expressing the nuclease. At least one additional sgRNA targeting the gene encoding the counter-selectable marker is introduced at the same time by the same mechanism as introduction of the guide molecule for the target nucleic acid thus coordinating introduction of both types of guides into the same cell. Thus, by selecting shoots having introduction of the guide targeting the gene encoding the counter-selectable marker, one can select for shoots also having the guide molecule for the target nucleic acid, allowing for selection of the desired cleavage event in the target nucleic acid. Said another way, the counter selection is applied to the plant such that the counter selection agent is delivered to the wound, thereby killing or reducing the growth of shoots containing the counter-selectable marker unless the gene for the counter-selectable marker has been altered by the nuclease as targeted by the at least one additional sgRNA targeting the gene encoding the counter-selectable marker. Accordingly, shoots generated from the wound, in the presence of the counter selection agent, will be enriched for those containing the altered counter selection gene and also the guide molecule for the targeted nucleic acid.

Any counter selection marker can be used as desired. In some embodiments the counter selectable marker itself is non-toxic to the plant, but converts an agent to a toxic molecule, if the counter selectable marker is active (i.e., has not been targeted by the nuclease). Exemplary non-limiting counter selectable markers and agent pairs include, D-amino acid oxidase and a D-amino acid (see, e.g., US20070016973), or Herpes Simplex Virus-1 Thymidine Kinase (HSVtk) and ganciclovir (see, e.g., Czakó M et al., Plant Physiol. 1994 March; 104(3):1067-71) or CodA mutated Escherichia coli cytosine deaminase (codA D314A) which converts nontoxic 5-fluorocytosine (5-FC), to 5-fluorouracilin, a pyrimidine that is incorporated into RNA during transcription and leads to cell death (Osakabe, K., et al., A mutated cytosine deaminase gene, codA (D314A), as an efficient negative selection marker for gene targeting in rice. Plant and Cell Physiology, 2014. 55 (3): p. 658-665).

Exemplary promoters for use in shoot meristem-specific expression include but are not limited to the Solanum lycopersicum LeT6 promoter (see, e.g., Uchida, Naoyuki, et al. Proceedings of the National Academy of Sciences 104.40 (2007):15953-15958)).

### Types of Plants

The methods described herein it is believed can be used on any plant species. In some embodiments, the plant is a dicot plant. In some embodiments the plant is a monocot plant. In some embodiments, the plant is a grass. In some embodiments, the plant is a cereal (e.g., including but not limited to Poaceae, e.g., rice, wheat, maize). In some embodiments, the plant is a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. In some embodiments, the plant is selected from the species: Brassica napus, Cucumis melo, Cucurbita pepo, Daucus carota, Gossypium hirsutum, Glycine max, Helianthus annuus, Linum usitatissimum, Papaver somniferum, Phaseolus vulgaris, Solanum lycopersicum, Spinacia oleracea, or Vigna unguiculata.

### EXAMPLES

### Background

A major challenge in genome editing is selecting of cell and cell lines what were mutated, to date no such selection method is available. This proposal aims to develop a protocol that allows us to easily identify and isolate tissues that have experienced high levels of CRISPR induced mutagenesis. Studies reported that genomic editing by CRISPR/Cas9 in one genomic site coincided with changes in another when several sgRNAs are used. We therefore suggest that a negative selection marker can be used to enhance the number of identified occurrences of successful activity of Cas9. To select for plants with edited genome we will generate lines with a gene that is conditionally lethal. In our case, we will engineer tomatoes with a marker gene encoding an enzyme that transforms a harmless chemical compound into a toxic one. CRISPR-mediated targeted co-mutagenesis of the at marker and a gene of interest will result in development of shoots resistant to the chemical and application of the compound will allow us to kill tissues that haven't been edited by Cas9.

### Transgenic Plants Expressing CAS9 and a Negative Selection Marker

In this study we used two independent selection markers, D-amino acid oxidase (DAAO) and Herpes Simplex Virus-1 Thymidine Kinase (HSVtk), which have been shown to be effective in plants. The D isomers amino acids (DAA) D-valine and D-isoleucine are not toxic to most organises including plants. However, plant engineered to express a non-native DAAO convert these compounds to ammonia and 2-oxo-carbon acids that are phytotoxic, therefore DAAO can also be used for low-cost (at ca. a ⅓ of the cost of the routinely used antibiotic kanamycin) negative selection. Viral HSVtk encodes an enzyme that converts the chemical ganciclovir, used to treat human viral infections, into ganciclovir triphosphate, which is toxic as it inhibits DNA synthesis.

We generated transgenic tomato plants co-expressing Cas9 under the control of the strong constitutive ubiquitin promoter and a counter selectable marker gene, doa1 from Rhodotorula gracilis and HSVtk, under the control of the specific shoot meristem STM promoter. These transgenes will be harmless to the plants as Cas9 is inactive without sgRNAs and DAAO and HSVtk do not produce phytotoxins in the absence of D-valine/D-isoleucine and ganciclovir, respectively. Because the marker genes are expressed just in meristems application of the selecting compounds will lead to death of only shoot meristems. T0 plants were selfed and T1 plant with single transgene insertions isolated. We selected the best preforming lines with the highest activity of transgene and used them to calibrate application counter selection, to further optimize agroinfiltration and shoot regeneration after decapitation.

### Transient Transformation and Selection for Mutations in the Negative Selection Marker

Once we selected the best preforming tomato lines with Cas9/DAAO and Cas9/HSVtk we used them for transient expression of sgRNAs and genome editing. Initially we tested two approaches for sgRNAs expression one based on Agrobacterium infection by vacuum infiltration, as we routinely do, and the second based on a viral expression system, using Tobacco Rattle Virus (TRV) or Potato Virus X (PVX), recently reported as applicable for CRISPR/Cas9. To test CRISPR and selection efficiency in our lines we cloned sgRNAs targeting DAAO or HSVtk under the control of the U6 promoter and terminator in Agrobacterium binary vector as we have done before [Ron et al., Plant Physiology, (2014) 114.]. We infiltrated Cas9/DAAO and Cas9/HSVtk plants with DAAO-sgRNA and HSVtk-sgRNA constructs, respectively, once the first internode is apparent on the seedlings.

### Experimental Description

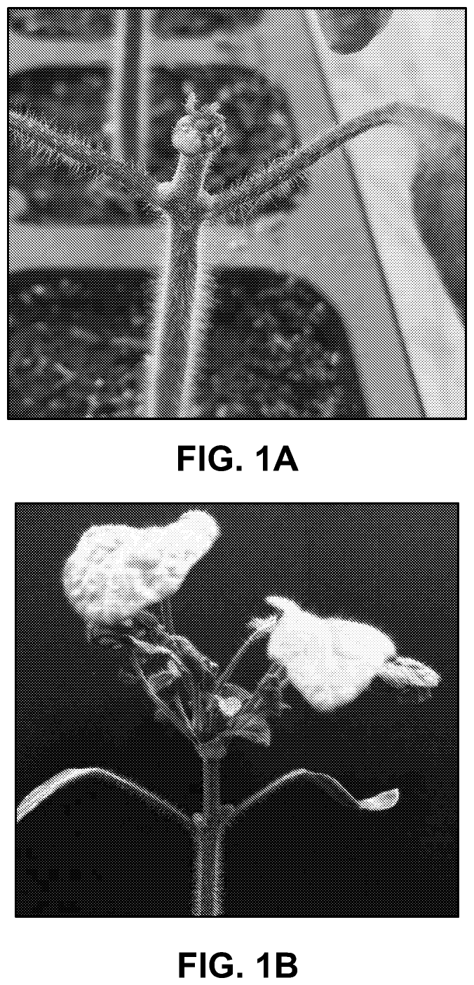

Angiosperm seedlings possess a high capacity for regeneration and will rapidly regenerate de-novo shoots upon decapitation. Hormonal signals promote de-differentiation of cells at the wound site and formation of a callus mass, which gives rise to de-novo formation of numerous shoot meristems. Within 30 days of seedling decapitation outgrowth of multiple de-novo shoots can be observed from each callus mass (FIG. 1A, B). The number of shoots arising from the callus can be increased more than 10 fold by shading of the cut stem during the regeneration process (Johkan et al, 2008). The number of bud-like meristems on each callus significantly outnumbers the shoots that will ultimately develop from the wound site. After the expansion of the first few shoots the remaining shoot buds halt their development suggesting apical dominance of the earliest expanded shoots. Removal of these shoots from the callus mass releases some of these buds which undergo expansion giving rise to additional shoots. A negative selectable marker system leading to selective ablation of shoot meristems not possessing the desired genome modification prevents inhibition of shoots derived from the genome modified cells, significantly increasing the likelihood of regenerating shoots with the intended modification.

In this study, we developed a CRISPR/Cas9-based plant gene editing method using counter selection against non-edited cells. We identified a series of potential counter selectable markers (CSM) that act as conditional lethal markers. They are non-lethal to plants in the absence of specific substrates, but become lethal when these substrates are delivered to the plants. The CSMs can turn the non-lethal agents into lethal chemicals, which inhibit plant growth, and eventually kill off the plant. In our study, the lethality of the CSMs was limited only to the apical meristem by using a specific promoter. The conditional lethal genes are expressed under the control of LeT6 promoter, which comes from Solanum lycopersicum LeT6 gene (Uchida, N., et al). The tomato LeT6 (Lycopersicon esculentum T6) gene is a class 1 knox gene, and is orthologous to the Arabidopsis stm1 (Shoot Meristem-less) (Chen, Ju-Jiun, et al). Knox genes are known to regulate plant development in many dimensions. Driven by the LeT6 promoter, the expression of the CSMs is specific to the apical meristem, which allows the supporting tissue of the plant to stay alive no matter what agent is applied.

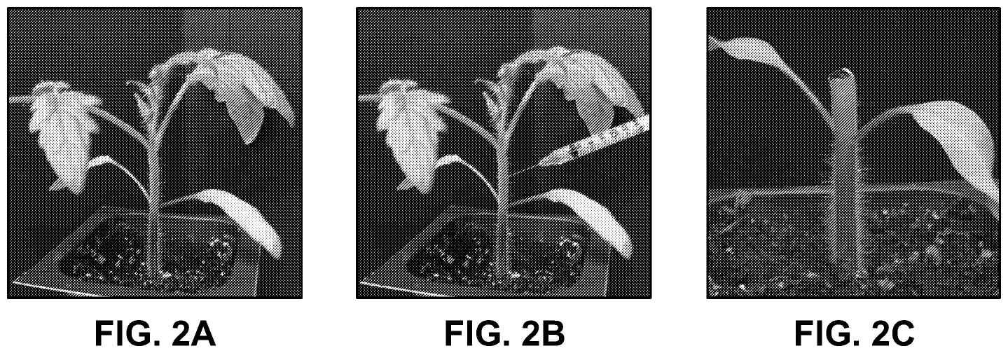

In one approach (FIG. 2), we first created a tomato transgenic line carrying a CSM driven by the LeT6 promoter. The plant also carries Cas9, but there are no gRNAs in the plant yet. Then we deliver the gRNAs targeting both the CSM and any gene(s) of interest based on the ability of CRISPR/Cas9 system to perform multiplex editing. The gRNAs are delivered by Agrobacterium injection into the shoot, just below the first pair of true leaves. After about five-seven days, the injected plants are decapitated at the injection site, letting new shoots regenerate from the wound site. Selecting agents are applied to the wound upon decapitation, which leads to meristem death in the non-mutated cells whose CSM is still functioning. On the other hand, cells in which the CSM sequence has been mutated can no longer be affected by the selecting agent, and can divide and differentiate to generate new shoots from the wound site. Meanwhile, chances are high that the co-expression of gRNAs targeting the gene(s) of interest at the same time as the CSM will result in mutation(s) in the desired gene(s). Therefore, by knocking out the CSM, we can select for the mutated new shoots, which can then produce seeds that are enriched in mutations in the gene(s) of interest.

### Counter Selectable Marker: DAAO

One potential CSM is DAAO (encoding D-amino acid oxidase), which originates from dao1 gene in yeast Rhodotorula gracilis and has been codon-optimized for tomato. The enzyme DAAO catalyzes the oxidative deamination of some D-amino acids (Alonso et al.). D-amino acid metabolism in plants is very restricted. Studies on Arabidopsis thaliana has shown that some D-amino acids, such as D-serine and D-alanine, can inhibit plant growth even at a low concentration, while other D-amino acids, such as D-valine and D-isoleucine have very little influence on plant growth (Erickson, O. et al.). However, when D-valine and D-isoleucine are metabolized by D-amino acid oxidase into keto acids, they become strongly toxic to plants. According to the Arabidopsis study, both D-valine and D-isoleucine, at the level of 30 mM, have deleterious effects on plants that express DAAO. Therefore, we employed DAAO as the potential conditional lethal marker with D-valine and D-isoleucine being the selecting agents.

### Counter Selectable Marker: HSVtk

Another CSM is HSVtk (encoding herpes simplex virus thymidine kinase type1), which has been used as a conditional lethal marker in mammalian cells [9]. The enzyme can phosphorylate nucleoside analogs, such as ganciclovir (GAN), into DNA replication inhibitors that are toxic to cells. Studies on Arabidopsis thaliana have shown that HSVtk can be used as a conditional selectable marker in plants as well (Czakó et al., 1994). Ganciclovir (GAN) is an antiviral drug. It can be metabolized by HSVtk and turned into a toxic form, which inhibits plant growth. According to Arabidopsis studies, 0.1 mM GAN can significantly reduce shoot regeneration on transgenic Arabidopsis root explants or callus formation on leaf explants, while it does not affect the regeneration of transgene-free explants.

Virus Vectors pTAV and pTRV

Geminiviridae is a family of plant viruses which have single-stranded circular DNA genomes and replicate via a rolling circle mechanism Hanley-Bowdoin et a., 2013). Studies have shown that efficient genome editing can be achieved using the geminivirus replicons in Arabidopsis and in tomatoes (Baltes, Nicholas J., et al, 2014,. Čermák, Tomáš, et al., 2015). In this study, we used a Begomovirus (a genus in the Geminiviridae family)-based DNA expression vector to carry the gRNAs. Begomovirus genomes are often bipartite, consisting of components A and B. The genome is a circular ssDNA, which replicates through double-stranded intermediates. The component A encodes five or six proteins: capsid protein (CP), replication-associated protein (Rep), transcriptional activator protein (TrAP), replication enhancer protein (REn), protein AC4, and protein AV2 in some strains. The component B encodes two proteins: movement protein (BC1) and nuclear shuttle protein (NSP), both are involved in movement of the virus within the infected plant. There is a stem-loop structure in the intergenic region that includes a conserved sequence (TAATATTAC) where ssDNA synthesis is initiated. The components A and B each have a common region (CR), which is an approximately 200 bp fragment in the intergenic region. There are also two divergent promoters within the common region, responsible for differential regulations of the expression of the viral genes. The replication of Begomovirus genome is initiated by the recognition of the common region. The ssDNA is converted to double-stranded by the host DNA polymerase and is amplified into many copies by rolling circle replication. Component B is dependent on A for replication. The vector pTAV that we are using was developed from the Tomato Mottle Virus (ToMoV), a species in the Begomovirus genus. The component B element and the capsid protein (CP) from component A are not present, therefore the virus cannot move from cell to cell. Once expressed in plant cells after Agrobacterium-mediated delivery of the DNA, the viral proteins Rep, TrAP and REn together with the plant DNA polymerase amplify the viral replicon sequence by rolling circle replication and lead to many copies of the gRNAs being produced.

We also used another virus vector developed from Tobacco Rattle Virus (TRV) to carry the gRNAs. TRV has been used as an efficient vector in virus-induced gene silencing (VIGS) in plants (Liu, Y., Schiff, M., and Dinesh-Kumar, S. P., 2002). It has also been reported in recent studies as a useful tool in facilitating CRISPR/Cas mediated genome editing in plants (Ali, Zahir, et al. 2015A+B). TRV has a bipartite genome, consisting of two single-stranded RNAs, RNA1 and RNA2. RNA1 encodes two replicase proteins, a movement protein and a cysteine-rich protein (Liu, Y., et al., 2002). RNA2 encodes the coat protein (CP) and two non-structural proteins. The non-structural genes in RNA2 can be replaced with a multiple cloning site for cloning the gene sequences for the gRNAs. TRV has been developed into a vector by cloning the cDNA of RNA1 and RNA2 into a T-DNA vector (Liu, Y., et al., 2002). The vector containing cDNA of RNA1 was named pTRV1, and the vector containing cDNA of RNA2 was named pTRV2 by Liu et. al (Liu, Y., et al., 2002). After agro-injection into the plant, transcription of the T-DNA will lead to the generation of RNA1 and RNA2 genomes of the virus. The two parts of the genome will lead to the generation of a whole virus capable of spreading throughout the plant. The two vectors pTRV1 and modified pTRV2 (e.g harboring sgRNA expressing cassette) were transformed separately into agrobacteria. The two Agrobacterium strains were simultaneously injected into tomato stems to deliver the gRNAs. However, the size limitation in the capacity of TRV (2-3 kb) prevents inclusion of the Cas9 gene. For this reason, the transgenic plants already carry Cas9.

### Tomato Leaf Developmental Gene: ENTIRE

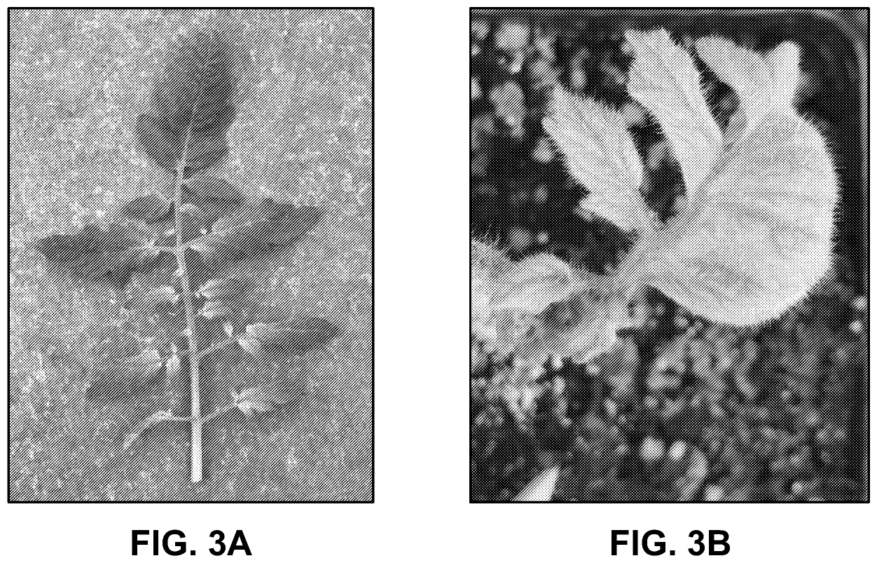

The tomato leaf developmental gene ENTIRE plays an important role in controlling leaf morphology. Mutations in ENTIRE lead to reduced complexity in tomato leaves. A wild-type tomato leaf is usually compound (FIG. 3A), while an entire mutant has fewer leaflets than a wild-type leaf (FIG. 3B), sometimes ending up having a large simple leaf. The plant hormone auxin is known to be involved in a lot of developmental processes in plants, from embryogenesis to fruit ripening. It also plays a role in leaf patterning, contributing to the development of compound leaves in tomatoes. Several studies have shown that the pleiotropic phenotype is due to loss-of-function mutations in the tomato AUX/IAA transcription factor IAA9 (Wang, H., et al., 2005, Zhang, J., et al., 2009). The AUX/IAA proteins can bind the Auxin Response Factors (ARFs), which are transcription factors that mediate auxin transcriptional responses, and then inhibit plant's response to auxin (Koenig, Daniel, 2009). However, with the presence of auxin, AUX/IAA proteins are degraded, and the response to auxin is activated. Studies have shown that ENTIRE can inhibit auxin-induced leaflet formation, and that the auxin-regulated degradation of ENTIRE contributes to appropriate compound leaf formation in early stages.

In this study, we included ENTIRE gene as one of the CRISPR targets that we aimed to knock out in tomato shoot meristems because of the overt leaf phenotype seen in plants carrying mutations at the ENTIRE locus. We hypothesized that the knock-out of ENTIRE can give us a phenotype of changes in leaf shape in the regenerated leaves.

### Tomato Leaf Developmental Gene: POTATO LEAF (C)

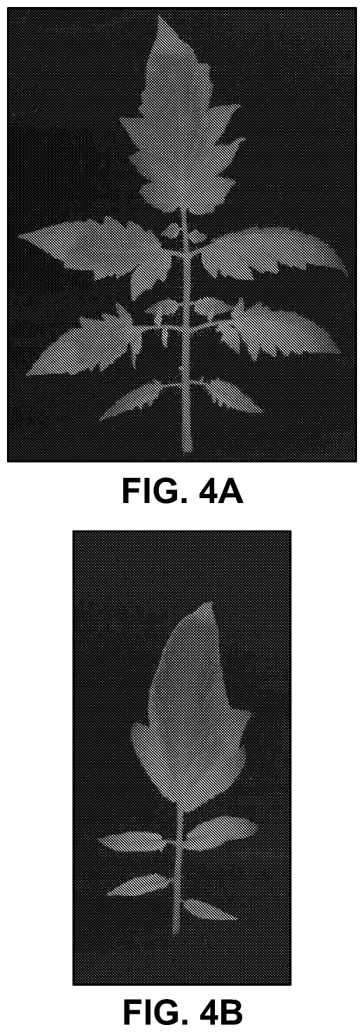

The tomato leaf developmental gene C plays an important role in controlling leaf morphology. Mutations in C lead to reduced complexity and reduced serrations in tomato leaves. A wild-type tomato leaf is usually compound (FIG. 4A), while a c mutant has fewer leaflets with smoother margins than a wild-type leaf (FIG. 4B). The C locus encodes a MYB-domain containing transcription factor (Koenig, D., et al., 2009) and many classic mutations exist at this locus that include insertions of retrotransposons, deletions and other alteration in the coding sequence.

In this study, we included C gene as one of the CRISPR targets that we aimed to knock out in tomato shoot meristems because of the overt leaf phenotype seen in plants carrying mutations at this locus. We hypothesized that the knock-out of C can give us a phenotype of changes in leaf shape in the regenerated leaves.

In example one, we used plants expressing the DAAO CSM. We decapitated the seedlings at the first internode so that cells that were genome edited could form a callus, regenerate new meristems and stems. The decapitation site was covered with parafilm and an aluminum foil cap for 4 weeks. Once callus formed we applied D-valine/D-isoleucine by spraying and/or irrigation (amino acids are uptaken by plant roots). Only meristems in which the selection marker DAAO was mutated and knocked out by Cas9/sgRNA can develop into healthy stems that will be detached and propagated. Mutation level and type were analyzed by PCR and sequencing of amplicons flanking the target genomic sites. We cloned sgRNA to mutate ENTIRE gene in tandem to DAAO-sgRNA using the tRNA processing approach. The Cas9 expressing mother plants were injected with viral replicon vectors and selection applied after decapitating. Regenerated stems were evaluated for mutation in the marker and ENTIRE sequences and the expected change in leaf shape typical of an ENTIRE knockout. Plants were propagated to fruiting and heritability of the phenotype and genotype was determined. Plants without Cas9 segregated from these T1 and showed heritable E mutant phenotypes.

In example two, we decapitated the seedlings at the first internode so that cells that were genome edited could form a callus, regenerate new meristems and stems. The Cas9 expressing mother plants were injected with the viral replicon vector and decapitated at the epicotyl a week later. The decapitation site was covered with parafilm and an aluminum foil cap for 4 weeks. Any axillary buds in the cotyledon node were removed. When a solid callus mass was visible (approx. four weeks after decapitation) at the wound site, the caps were removed and ganciclovir was applied (2 mM concentration of the compound in a carbomer gel). Every meristem that developed into a healthy shoot was detached and propagated to fruiting. Mutation level and type were analyzed by PCR and sequencing of amplicons flanking the target genomic sites. Regenerated stems were evaluated for mutation in the marker and POTATO LEAF (C) sequences and the expected change in leaf shape typical of a C knockout. We evaluated the correlation between the Cas9 modified HSVtk and C.

### Materials and Methods

### Plant Material

We used tomato cultivar M82 as the wildtype background. Seeds were obtained from plants grown in the fields in Davis, Calif. Three T₀ transgenic lines were used; DAAO and Cas9, HSVtk transgenic lines, and HSVtk and Cas9 lines in which the CSM was driven by the LeT6 promoter. They were generated by the UC Davis transformation facility. The T₀ plants were propagated to produce seeds, and the T₁ plants were used in the experiments described below.

### Constructs and Primers

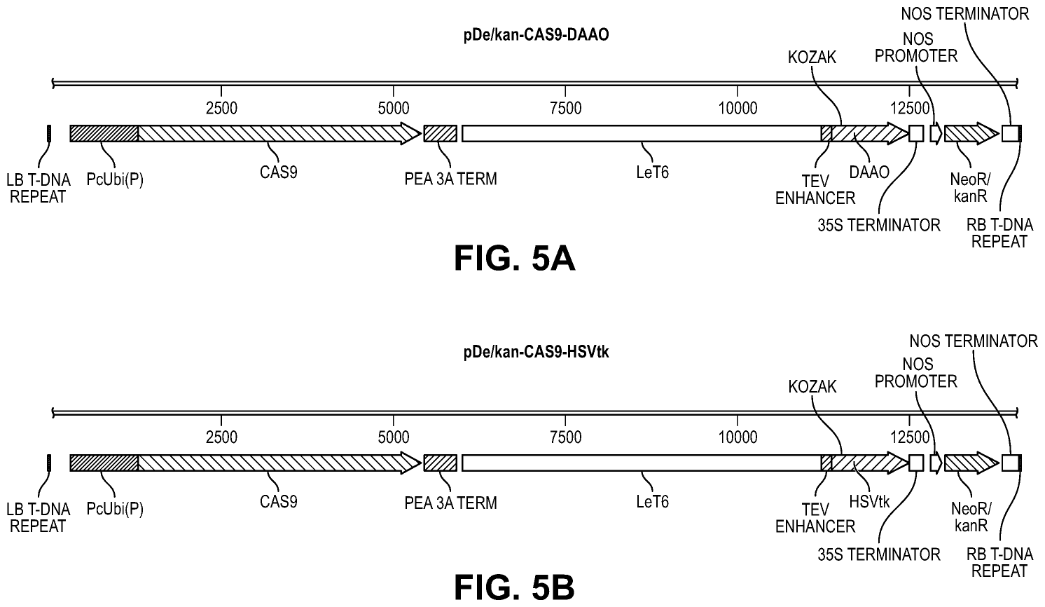

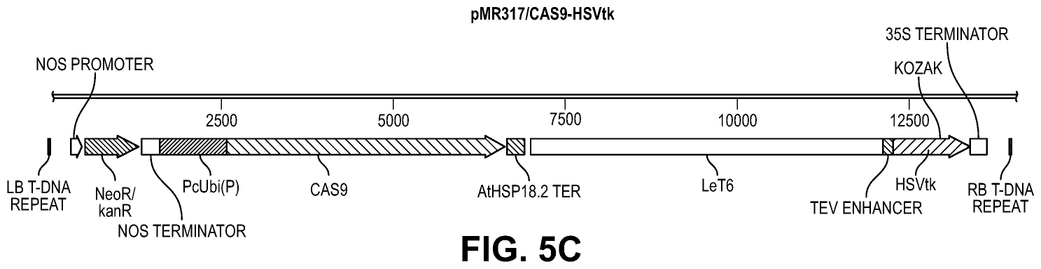

The DAAO-Cas9 transgenic plants were transformed with the construct (pDe/Kan-Cas9-DAAO) consisting a codon-optimized S. pyogenes Cas9 under the control of the parsley ubiquitin promoter (PcUbi), a synthetic tomato codon-optimized DAAO under the control of LeT6 promoter (LeT6p), and NPTII cassette under the nopaline synthase (NOS) promoter for resistance to kanamycin, neomycin and G418 (FIG. 5A). The codon optimized DAAO was generated using IDT web tool based on tomato codon usage and to select a sequence without splice sites. The primer pair SIDAAO-F+501 (AGCTCTGAATGTCCACCAGGAGC (SEQ ID NO: 1)) and SIDAAO-R+754 (ACCAATACAGTTTGCCCTCGGA (SEQ ID NO: 2)) were used in genotyping DAAO in DAAO T₁ plants which were germinated in soil. The primer pair LeT6proend (CAGTGTGTGTGAGAGAGAGAGATGG (SEQ ID NO: 3)) and SIDAAO-R+754, which flanked the gRNA target regions within DAAO, were used to test CRISPR introduced mutations in DAAO in DAAO plants that were injected with gRNAs. The primer pair ENT.FOR2 (GAGGAGGGCCAGAGTAATGT (SEQ ID NO: 4)) and ENT.REV2 (GTGGCCAACCAACAACCTGT (SEQ ID NO: 5)) were used to test CRISPR introduced mutations in ENTIRE in the DAAO plants.

The HSVtk-Cas9 transgenic plants were transformed with the construct (pDe/Kan-Cas9-HSVtk) consisting of the same codon-optimized S. pyogenes Cas9 under the control of the parsley ubiquitin promoter (PcUbi), a tomato codon-optimized HSVtk under the control of LeT6 promoter (LeT6p), and NPTII cassette under the nopaline synthase (NOS) promoter for resistance to kanamycin, neomycin and G418 (FIG. 5B). However, the T0 plants generated with this construct had truncated T-DNA inserted and contained only the Let6pro-HSVtk expression cassette with no CAS9. These plants were used to test the sensitivity of the plants to the ganciclovir agent. The primer pair SIHSVtk-F+527 5′-AATGGGTATGCCATACGCTGTTAC-3′ (SEQ ID NO: 6) and SIHSVtk-R+726 5′-TAAGAGCGACGAAAGCCAAAAC-3′ (SEQ ID NO: 7) were used to genotype HSVtk in HSVtk T₁ plants that were germinated in soil. In order to generate transgenic plants that express both the CAS9 and the HSVtk cassette we generated a new vector, pMR317 in which the selectable marker was transferred close to the T-DNA LB (FIG. 5C).

Virus Based Vectors pTAV and pTRV

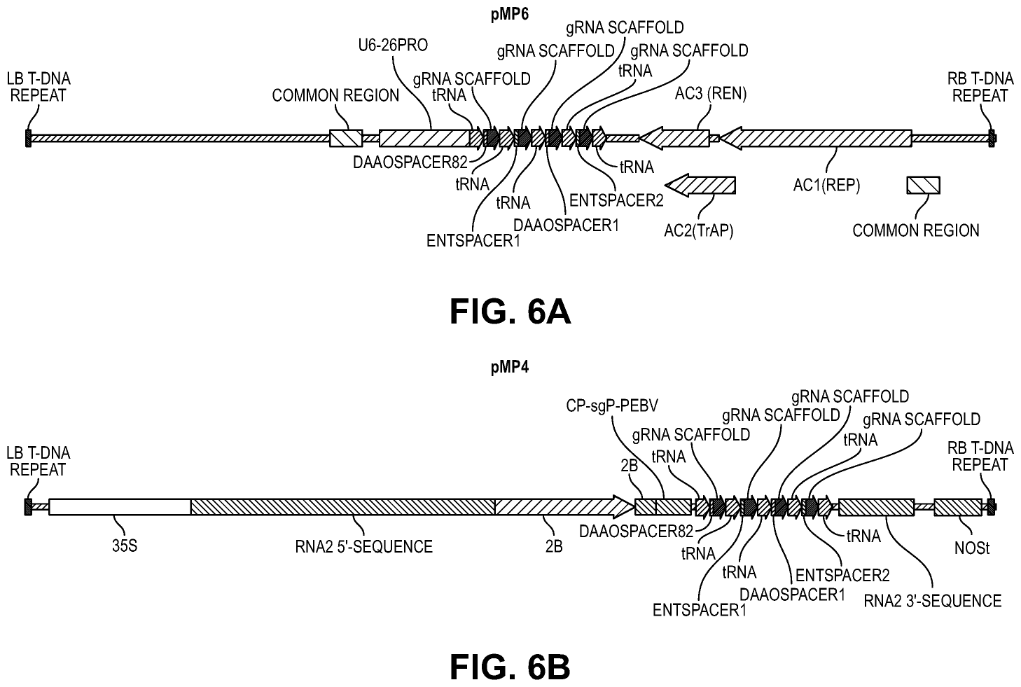

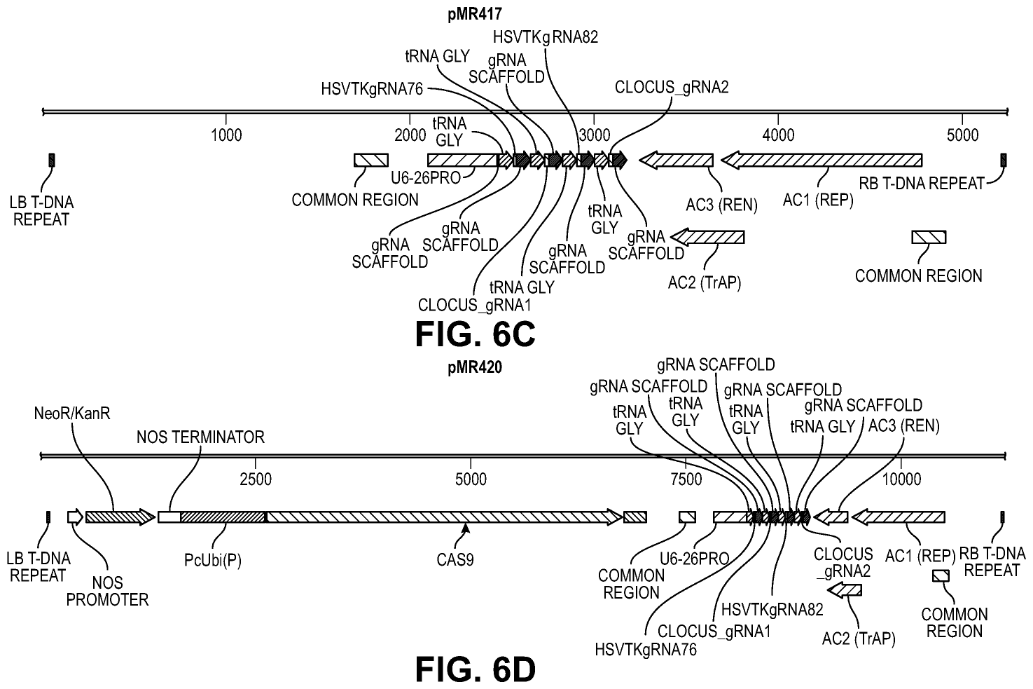

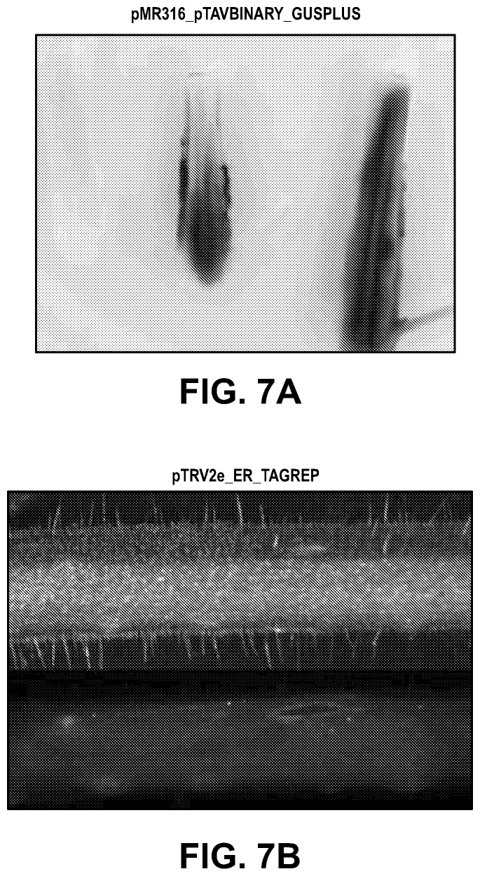

The begomovirus vector pTAV was modified for Agrobacterium injection and GATEWAY cloning, which became pMR315 (pTAV-GW). The tRNA-gRNA structure with DAAO spacers and ENTIRE spacers was synthesized and cloned into pEn_Chimera followed by an LR recombination into the binary vector pMR315 to generate pMP6 (FIG. 6A). The vector was transformed into Agrobacterium strain AGL1, and the transformation was verified by colony PCR. The complete sequence of pMP6 is in the appendix. The Binary vector pMR315 has the common regions for recognition and three ToMoV proteins: Rep, TrAP and REn. The tRNA-gRNA architecture carrying spacers for DAAO and ENTIRE is driven by U6 promoter. Each spacer is flanked by a tRNA and a gRNA scaffold. There are two spacers for each gene. The neomycin/kanamycin resistance gene is included for future plant selection purposes. T7 and SP6 promoters are included so that RNA can be synthesized from both strands of the insert DNA.

pTRV1 (pYL192) was from the Dinesh Kumar Lab (University of California, Davis). Its sequence and map can be found in supplement sequence 7 in (Ali, Z., et al. 2015).

pTRV2 (pYL156) was also from Dinesh Kumar Lab. It was modified and renamed it as pTRV2e. The tRNA-gRNA construct with DAAO spacers and ENTIRE spacers was cloned into pTRV2e, by restriction/ligation to generate pMP4 (FIG. 6B). pMP4 was transformed into Agrobacterium strain AGL1, and the transformation was verified by colony PCR. The complete sequence of pMP4 is in the appendix. It has a 2b gene, which is involved in transmission of the virus in TRV strain Ppk20 (Vassilakos, N., et al., 2001), followed by the same tRNA-gRNA structure described above, which is enhanced by CP-sgP-PEBV, with PEBV promoter (the promoter of Pea early browning virus). The RNA2 5′-sequence and RNA2 3′-sequence of TRV strain Ppk20 are flanking the 2b and tRNA-gRNA structure, under the control of CaMV 35S promoter, terminated by a NOS terminator.

generate pMR420 (FIG. 6C) and the binary vector pMR410 that contain also a CAS9 expression cassette to generate pMR417 (FIG. 6D).

### Agrobacteria Injection

Agrobacterium glycerol stocks transformed with pTRV or pTAV vectors were streaked onto LB plates containing appropriate antibiotics based on plasmids and agro strains. Plates were placed in 30° C. room for three days to allow for growth of the bacteria. Streaks were taken from these plates, and added to 10 mL of LB containing antibiotics in a 50 mL falcon tube. Falcon tubes were put on shaker at 200 rpm for 24 hours in 30 C room. After 24 hours, cultures were measured for OD600 using spectrophotometer. If OD600 was 1.500 or above, 1 ml of LB culture was added to 9 mL Induction Media (autoclaved before use) containing antibiotics and 200 uM acetosyringone (ACS). If OD600 was below 1.500, 2 mL of LB culture was added to 8 mL of Induction Media. Induction Media cultures were grown in 50 mL Falcon tubes on 200 rpm shaker in 30 C room for 24 hours. The next day, OD600 was measured for each culture. Falcon tubes were centrifuged at 3000rcf for 10 minutes. Liquid was decanted from the tubes, and pellet was washed with sterilized Reverse osmosis (RO) water. Pellet was resuspended to an OD600 of 1.000 in filter-sterilized Inoculation Buffer containing 200 uM ACS. Tubes were placed on shaker at 150 rpm in 23 C room for 3-6 hours. After removal from shaker, 0.5 mM dithiothreitol (DTT) was added to Inoculation Buffer.

Tomato seedlings, 2-3 weeks old, were well irrigated the morning of infiltration. Agrobacterium in Inoculation Buffer was injected into the stems of the seedlings using a 12 mL Monoject syringe with a 30G needle. Seedlings were injected 2 cm above cotyledons, in the first internode of the plant. The needle was inserted at an upward angle, roughly 5 mm into the stem and the syringe plunger was depressed until there was too much resistance to inject any more. This was repeated twice more around the stem, at two other areas 2 cm above the cotyledons. Seedlings were placed in 16 hour light/8 hour dark growth chamber at room temperature for 5-7 days to allow gRNAs to be expressed.

### Decapitation

Roughly one week later, seedlings were decapitated at the injection site using a sharp razor blade. Immediately after decapitation, parafilm was stretched over the decapitation site, to prevent the stem from drying out, and aluminum foil was added to cover the parafilm, shading the cut site to promote callus formation. Plants were returned to growth chamber and monitored for axillary shoot formation and callus regeneration. New shoots forming from the cotyledon axillary buds were removed using forceps as soon as they were observed.

### Application of Counter-Selective Agent and Shoot Regeneration

After 1-2 weeks, the cut site of the stem began forming a white callus, and selection gel was applied. Carbomer 940 powder was added to a water solution containing selection agents to make 0.5% w/v carbomer gel. The pH was adjusted with KOH to 7.5 to thicken the gel. The gel containing the selecting agents was added to the decapitated plants by putting a droplet of the gel to the wound site of the shoot using spatula, about 40-50 uL in volume. Parafilm and aluminum foil was placed back on the cut site, and plants were returned to growth chamber. Roughly one month after decapitation, small shoots were observed to be growing from calli. Parafilm and foil were removed from decapitation sites, and shoots were allowed to grow.

Solutions of D-amino acids were made from D-valine powder (MP Biomedicals 0210322625) and D/L-isoleucine powder (MP Biomedicals 0210208225). Zirgan (ganciclovir ophthalmic gel) 0.15%, an antiviral eye gel was used in making GAN gel, as well as in direct application on decapitated plants.

When the shoots produced 2 nodes of true leaves, shoots were removed from the callus using razor blade. The bottoms of the shoots were dipped in Clonex rooting gel containing IBA, and the shoots were placed in wet jiffy rooting cubes. After 2-3 weeks, strong roots were established by the cuttings, and the jiffy cubes were planted in soil pots. Seedlings were transferred to greenhouse and grown for seed.

### DNA Extraction and Genotyping by PCR

Plant tissues such as leaves and meristems were collected (5-100 mg tissue in each tube, though more tissue usually results in more DNA yield) and frozen in liquid nitrogen. The frozen tissues were ground for 1 min using the Mini-Beadbeater (BioSpec Products) coupled with 4-6 silica beads (2.3 mm dia. ZIRCONIA/SILICA, BioSpec Products) in each tube of plant tissues. The ground tissues were put in standard CTAB buffer (3004 in each tube) and ground for another 1 min before being put for incubation at 65° C. for 15 min. After the incubation, chloroform/isoamyl alcohol (24:1) was added and the mix was centrifuged. Isopropanol was added to the supernatant to precipitate DNA. After some washing steps, DNA was eluted in the elution buffer. PCRs were conducted for both gRNA targets, and PCR products were sequenced using Sanger sequencing and analyzed for mutations.

### Poly-A RNA Extraction

Poly-A RNA was extracted through the protocol developed by B. Townsley [21] using NEB Streptavidin magnetic beads (Biolab, Cat. S1420S) and Biotin-linker-polyT oligo. The procedures involved stabilizing RNA in Lysis/binding buffer, capturing biotin-poly-dT-annealed RNA lysate with the magnetic streptavidin beads. After several washing steps, poly-A RNA was eluted in the elution buffer.

### Reverse Transcription PCR

The extracted mRNA was treated with DNase (RQ1 RNase-Free DNase, Cat. M6101, Promega), 1-84 RNA in elution buffer with 1 μL RQ1 DNase (the volume was brought to 10 μL by nuclease-free water), to eliminate genomic DNA before doing the reverse transcription PCR. The DNase treated mRNA was then used in first strand cDNA synthesis with the RevertAid First Strand cDNA Synthesis Kit from Thermo Scientific.

### TA Cloning

TA cloning of the PCR products were performed using the Invitrogen TOPO TA Cloning Kit. The cloning reactions were transformed into E. coli DH5a competent cells by heat shock and let to grow until colonies appeared.

### Results

### Toxicity of D-Valine and D-Isoleucine to DAAO Transgenic Plants

Before testing the toxicity of D-Val and D-Ile to DAAO transgenic plants, wildtype M82 plants were tested for their sensitivity to the two D-amino acids (DAAs). Three-week-old M82 plants grown on jiffy-7, 42 mm peat pellets (Manufacturer: Root Naturally) that offer quick rooting, were watered with tap water containing the DAAs, with different concentrations of DAAs ranging from 0 mM to 45 mM D-Val plus 60 mM DL-Ile. After six days, during which the plants were re-watered with only DAAs solutions from time to time, they were not affected at the concentration up to 30 mM D-Val plus 40 mM DL-Ile. At DAAs concentration higher than 30 mM D-Val plus 40 mM DL Ile, the M82 plants seemed to suffer, with leaves becoming withered and growing a little yellowish, in contrast to the healthy plants in the lower DAAs concentrations. Thus, we decided that 30 mM D-Val plus 40 mM DL-Ile was the DAAs concentration could be applied to soil-grown plants via different methods described later without causing a negative effect on wildtype plants. Carbomer 940 was chosen to make the gel to apply D-amino acids in our experiments due to its high viscosity (40,000-60,000 cps in 0.5% solution, pH7.5) and good clarity when dissolved in water. The powder of Carbomer 940 was dissolved in DAAs solution, the pH was adjusted to 7.5 to thicken the gel, and applied in 40 to 50 ul volume droplets to the cut site. The Carbomer gel without DAAs was tested on wildtype (M82) plants to make sure it did not influence plant regeneration. When treated with DAAs, these plants almost all regenerated new shoots. We tested 2 different concentrations of DAAs for selection (30 mM D-Val plus 40 mM DL-Ile or 60 mM D-Val plus 80 mM DL-Ile). In the former there was no effect on the DAAO transgenic plants and shoots regenerated at same rate as in M82. In the higher concentration both genotypes developed necrosis at the cut site and no callus was formed. Therefore, selection by DAA was not very effective in tomato using these concentrations and application methods, and may need further optimization.

### Knocking Out DAAO and ENTIRE

We introduced two gRNAs targeting DAAO, whose sequences were 5′-TGTGGTGGTGCTCGGTTC-3′ (SEQ ID NO: 8) and 5′-GACCAAGACAGGCCAAAT-3′ (SEQ ID NO: 9). At the same time, we also introduced two gRNAs targeting the tomato leaf developmental gene, ENTIRE, which would create changes in leaf shape if mutated. From our knowledge in a whole plant entire mutant, if the gene is mutated, in plants homozygous for the mutation [18] the leaf will fail to develop multiple leaflets and end up as a simple large leaf or a leaf with reduced complexity. Therefore, we hypothesized that if ENTIRE is mutated in the meristem, there will be an obvious phenotype in the regenerated leaves. The two gRNAs introduced to target ENTIRE were 5′-GGATTAAATCTCAAGGCAA-3′ (SEQ ID NO: 10) and 5′-GGATCTCAGTCTCCCGAAAG-3′ (SEQ ID NO: 11). The four gRNAs together were carried in the same vector through the tRNA processing approach, which allowed the possibility of multiplex editing.

We introduced two gRNAs targeting DAAO, whose sequences were 5′-TGTGGTGGTGCTCGGTTC-3′ and 5′-GACCAAGACAGGCCAAAT-3′. At the same time, we also introduced two gRNAs targeting the tomato leaf developmental gene, ENTIRE, which would create changes in leaf shape if mutated. From our knowledge in a whole plant entire mutant, if the gene is mutated, in plants homozygous for the mutation [18] the leaf will fail to develop multiple leaflets and end up as a simple large leaf or a leaf with reduced complexity. Therefore, we hypothesized that if ENTIRE is mutated in the meristem, there will be an obvious phenotype in the regenerated leaves. The two gRNAs introduced to target ENTIRE were 5′-GGATTAAATCTCAAGGCAA-3′ and 5′-GGATCTCAGTCTCCCGAAAG-3′. The four gRNAs together were carried in the same vector through the tRNA processing approach, which allowed the possibility of multiplex editing.

After injecting the agrobacteria carrying the gRNAs into the DAAO plants, we let the plants grow for 5-7 days, followed by decapitation. After about a month, both agro-injected and control decapitated plants formed calli and later regenerated new shoots from the cut site. No selection was applied on these plants

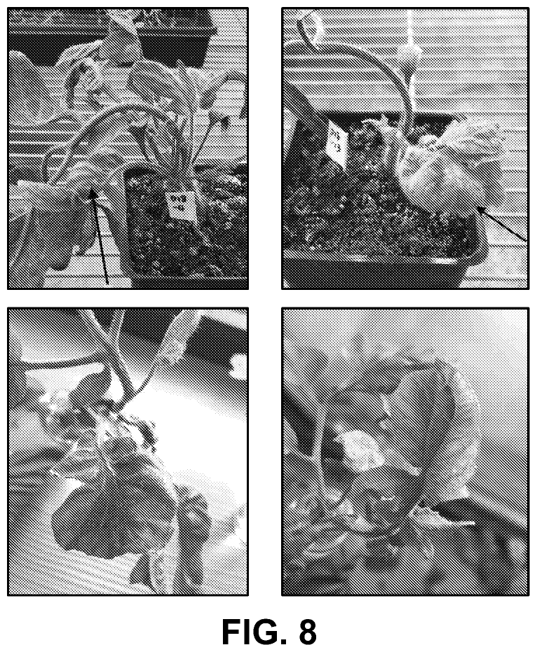

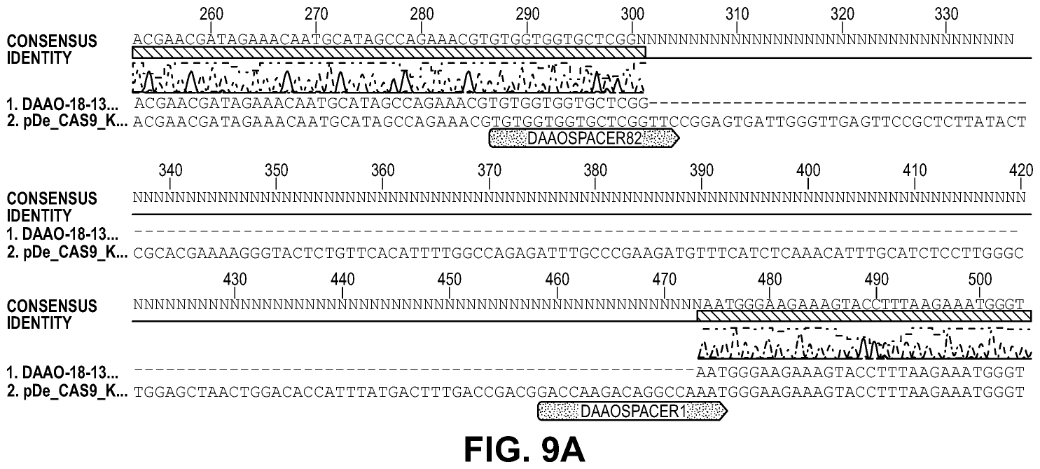

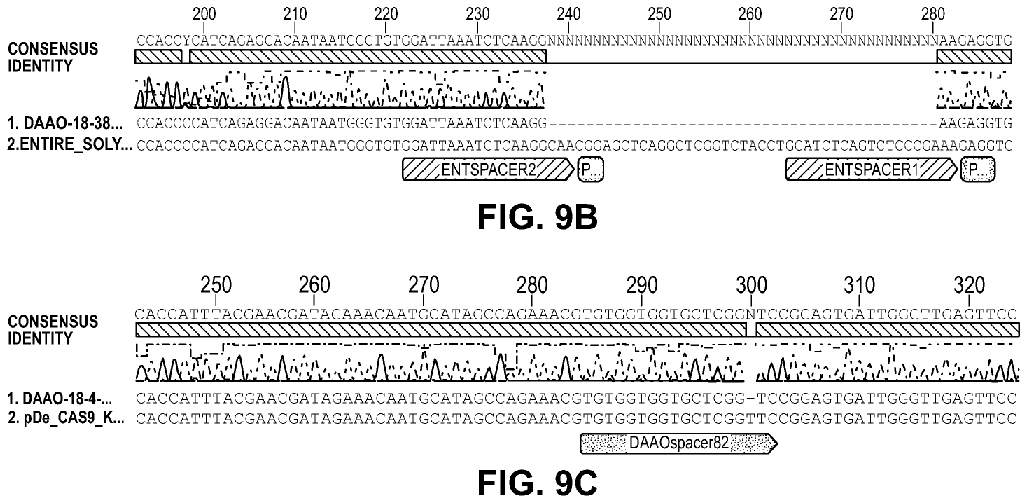

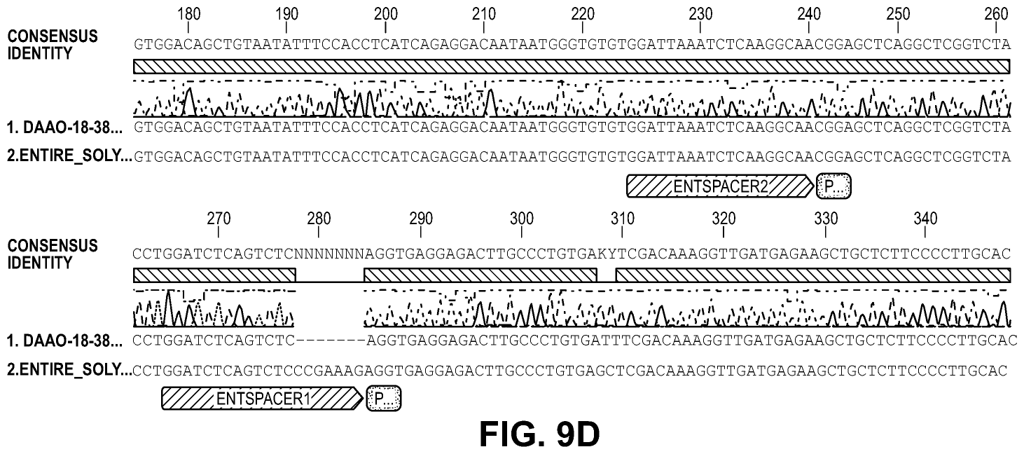

Considering the ENTIRE gene, we identified candidates which, in regenerated shoots looked like they carried a mutated ENTIRE locus (FIG. 8). The leaves on these shoots developed fewer leaflets than usual, consistent with the ENTIRE-mutated phenotype. Tissue from the candidate leaves was used for DNA extraction, PCR amplification, and Sanger sequencing of the DAAO transgene as well as the endogenous ENTIRE locus. We saw mixed peaks in the sequencing data suggesting chimeric editing. One out of six sequenced samples for DAAO produced mixed peaks, while two out of six for ENTIRE produced mixed peaks. We cloned the PCR products from all the samples for DAAO and ENTIRE into E. coli by TA cloning, and then sequenced the PCR product of each bacterial colony grown on the plate to get clean sequence data. Mutations were detected in DAAO in two (FIGS. 9A and C) of the cloned samples. One of the two had produced mixed peaks before. The other did not, possibly because it was a point mutation. Also, there were mutations detected in ENTIRE in the same two samples, and they were the same mutation (FIG. 9D). In another experiment, no mixed peaks were produced in DAAO, but there were two out of nine samples showing smaller bands than the regular size of DAAO on the agarose gel of PCR products. There were mutations in DAAO detected in these two samples (same mutation as shown in FIG. 9A). In the same experiment, three out of the nine samples produced mixed peaks in ENTIRE, but have not yet been cloned for Sanger sequencing. One sample (not producing mixed peaks) out of the nine showed a smaller band than the regular size of ENTIRE on the gel of its PCR product. It was sequenced and a mutation in ENTIRE was detected (FIG. 9B). Among the 15 entire candidates that we sequenced, three had mutations detected in both DAAO and ENTIRE, and one had a mutation detected in DAAO alone. Two of the candidates that had mutations in both DAAO and ENTIRE were found to have homozygous mutations in both genes, suggesting that they came from the same editing event. There was a 173 bp deletion between the two gRNAs in DAAO, and there was a 43 bp deletion between the two gRNAs in ENTIRE (FIG. 9A, B). The other double-mutated candidate had a 7 bp deletion in one of the gRNAs in ENTIRE (FIG. 9D), while it had several different mutations detected in DAAO, with one example shown in FIG. 9C. The one candidate that only had a mutation detected in DAAO had the same 173 bp deletion at the same location as the other two.

This experiment indicates that even in the absence of effective DAA selection, editing at two loci was easily achieved using this method.

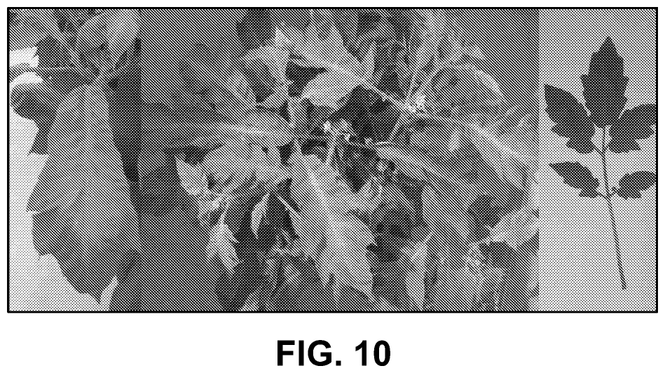

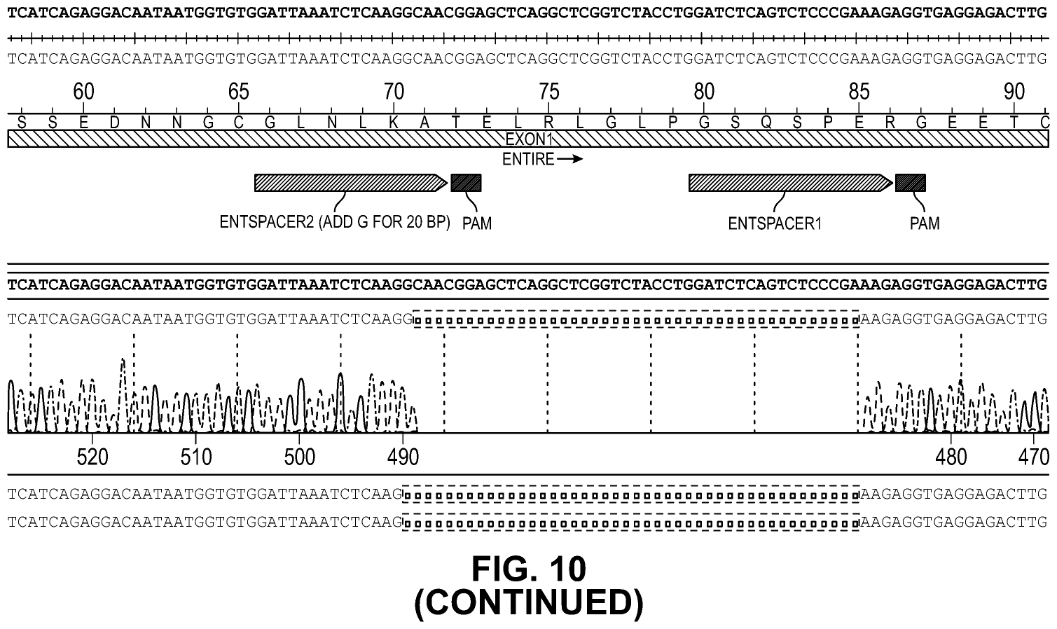

Three shoots carrying the entire mutation were rooted, and transferred to soil to encourage growth and reproduction. The progeny of these plants inherited the edited gene and the phenotype (FIG. 10).

### Toxicity of Ganciclovir to HSVtk Plants

HSVtk (no Cas9) plants were germinated and grown in soil. They were decapitated about three weeks after they were sown. The selecting agent, GAN (Ganciclovir), was applied to the decapitated plants after the decapitation. We made a series of GAN gels with different GAN concentrations: 0.1 mM, 1 mM, and 4.5 mM. The 0.1 mM and 1 mM GAN gel was made by diluting the Zirgan ganciclovir ophthalmic gel (4.5 mM GAN) into 0.2% carbomer gel. The two gels were mixed in a 4 mL plastic vial by shaking. Around 454 GAN gel was applied to each plant each time, and the gel application was renewed every three to five days.

We tested whether the carbomer gel itself would have an influence on plant regeneration. We included a group of wildtype plants that received no treatment, and a group of wildtype plants that received carbomer gel containing no GAN. All plants in both groups, except one plant in the control set, regenerated new shoots. Therefore, we eliminated the possibility of regeneration interference from the gel.

One month after the decapitation and application of GAN selection, the regeneration of shoots was evaluated. The results are listed in Table 3. In the control plants with 0 mM GAN treatment, including HSVtk and wildtype plants, almost all the plants regenerated new shoots. As the GAN concentration increased, both HSVtk plants and wildtype plants displayed decreased regeneration. When the GAN concentration reached 4.5 mM, none of the HSVtk plants regenerated, while still 6 out of 23 wildtype plants regenerated. Thus, we concluded that GAN was toxic to both HSVtk plants and wildtype plants at a concentration higher than 0.1 mM. However, as the GAN concentration increased, HSVtk plants became more sensitive to it than wildtype plants. At a concentration of 4.5 mM, GAN could prevent regeneration of the HSVtk plants, while still allowing some wildtype plants to regenerate. Therefore we decided to use a 2 mM dose as the effective selecting agent in our future experiments.

### Knocking Out HSVtk and C

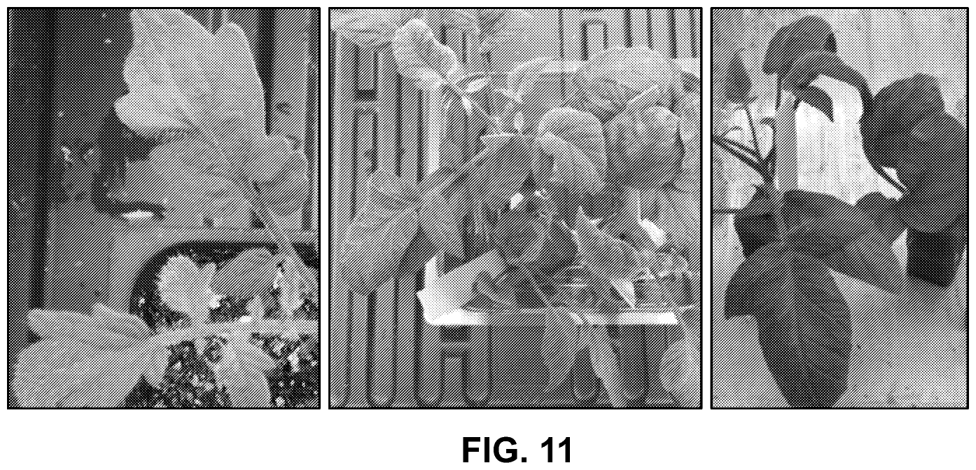

Viral HSVtk encodes an enzyme that converts the chemical ganciclovir, used to treat human viral infections, into ganciclovir triphosphate, which is toxic as it inhibits DNA synthesis (Czakó et al., 1995; Czakó & Márton, 1994). We tested this system extensively in tomato and found it useful as a CSM, as there are concentrations at which HSVtk+Cas9 transgenic lines showed suppression of shoot regeneration while wild-type tomato lines still showed some shoot regeneration. Therefore, as this marker is somewhat efficient at selecting for edits at the inserted HSVtk gene, we used these lines in experiments to transiently deliver guide RNAs and look for editing in target sites. HSVTk+Cas9 transgenic lines were injected with viral vectors containing the HSVtk and C-locus gRNAs without or with an additional Cas9 cassette in the vector. We applied 2 mM concentration of the compound ganciclovir in a carbomer gel on decapitated shoots to select against presence of the functional HSVtk transgene. We tested all the shoots that were regenerated for mutations. Out of all 33 regenerated shoots, 7 had the potato leaf phenotype (presuming homozygous or biallelic mutations—Table 2, FIG. 11). In these same 33 shoots, 11 were mutated at HSVtk (˜33% efficiency—Table 2). Four homozygous HSVtk mutant shoots also showed the potato leaf phenotype. Our data suggests that adding extra Cas9 (in addition to that already expressed in the transgenic injected plant) can boost CRISPR efficiency for editing (Table 2). Two of the HSVtk mutant plants carry heterozygous lesions at C and thus do not show the c phenotype. As a comparison, unselected plants regenerate between 2-3 shoots per decapitated apex. In one preliminary test in an experiment with no selection and no visible phenotypic expectation at the AN3 locus, none of the ˜50 regenerated shoots tested carried mutations at the target site. Thus, our current scheme of HSVtk selection reduces the number of shoots to be tested, and conditions enrichment for CRISPR mutagenesis at target sites.

TABLE 2

| | | | | | | shoots with | shoots with | shoots with |
|---|---|---|---|---|---|---|---|---|
| | Number of | Number of | shoots | shoots with | shoots with | homozygous | heterozygous | mutations |
| | plants | regenerated | with c | homozygous | heterozygous | mutation at | mutation at | in both C |
| Construct | injected | shoots | phenotype | mutation at C | mutation at C | HSVtk | HSVtk | and HSVtk |
| pTAV.gRNA.CAS9 | 36 | 16 | 5 (31.3%) | 5 (31.3%) | 1 (6.3%) | 7 (43.8%) | 0 | 4 (25%) |
| pTAV.gRNA.CAS9 | 36 | 17 | 2 (11.8%) | 2 (11.8%) | 1 (5.9%) | 4 (23.5%) | 0 | 2 (11.8%) |

### Discussion

II. Transient Transformation and Selection for Mutations in the Negative Selection Marker

Four trials were conducted using the pTRV and pTAV viral vectors. Two guides for DAAO and two guides for ENTIRE (E, mutations lead to visible leaf phenotypes—(Koenig, Bayer, Kang, Kuhlemeier, & Sinha, 2009) were inserted into both vectors (Xie, Minkenberg, & Yang, 2015). Selection was applied to the cut site in 2/4 experiments. We did not see any difference in the number of shoots regenerated from WT or DAAO transgenic plants after the DAA application. Nevertheless, a subset of regenerated stems were evaluated for mutation in the DAAO transgene and ENTIRE sequences and the expected change in leaf shape typical of the entire knockout (FIG. 13—Koenig et al., 2009). In the 2 trials without selection, plants were injected with either TRV or TAV and 15 plants regenerated shoots with possible perturbations in leaf development reminiscent of entire leaf phenotypes. These 15 plants were genotyped for mutations.

III. Efficiency of the System as Tested in Tomato

Among the 15 entire candidates that we sequenced, three had mutations in both DAAO and ENTIRE, and one had a mutation in DAAO alone. One shoot was heterozygous for a 173 bp precise deletion between the two expected cut sites in DAAO, and also heterozygous for a 43 bp precise deletion spanning the two expected cut sites in ENTIRE. Another shoot contained the same mutations in DAAO and E, but in this instance was homozygous for the precise expected deletion in E. The third shoot had a 7 bp deletion at one of the gRNA target sites in E, while it was chimeric for DAAO based on assaying multiple leaves on the shoot. The one candidate that had a mutation detected in DAAO, but not E, was homozygous for the precise 173 bp deletion between the two gRNAs. All these mutated plants had been injected with the TRV construct.

TABLE 3
Combined Numbers for all Four DAAO/ENTIRE Injection Trials

| | | Shoots | Mutations | DAAO | ENTIRE |
|---|---|---|---|---|---|
| | Plants | tested for | in both | only | only |
| Construct | injected | mutation | genes | mutation | mutation |
| pTAV | 48 | 19 | 2 | 0 | 0 |
| pTRV | 49 | 18 | 4 | 2 | 1 |

In the 2 trials with selection, 56 plants were injected with TRV or TAV. Twenty-two regenerated shoots that hinted at early leaf development perturbations were sequenced for mutations. One shoot, from TAV injection, contained a homozygous 1 bp insertion in DAAO and a large 44 bp heterozygous deletion in E. Another plant injected with TAV contained a 3 bp heterozygous deletion in DAAO and a heterozygous 1 bp deletion in E. One plant injected with TRV contained a 1 bp heterozygous deletion in DAAO and a homozygous 44 bp deletion in E. Furthermore, one plant injected with TRV contained a heterozygous 4 bp deletion only in DAAO, and another plant injected with TRV contained a heterozygous 40 bp deletion only in E (Table 2). These results are an indication that early leaf phenotypes may either be due to chimeric lineages making up part of the shoot, or the regeneration process causing perturbations in early leaf development.

Shoots containing mutations in both genes were rooted and grown for seed. The T1 seeds were planted, and seedlings were sequenced to confirm heritability of mutations. Four out of 12 progeny from a heterozygous double knockout displayed the entire phenotype, while the others displayed a normal leaf phenotype. The four entire plants were found to have homozygous mutations in entire and heterozygous mutations in DAAO, while 5 normal looking plants contained heterozygous mutations in both DAAO and ENTIRE. All progeny from the homozygous entire knockout displayed the entire phenotype. These plants were sequenced, and all contained homozygous deletions in entire and segregated for mutations in DAAO. These results are an indication that even in the absence of efficient counter-selection, injection with viral vectors coupled with shoot decapitation is efficacious in CRISPR mutagenesis at two loci. Despite some phenotypic selection bias, the identified mutation rates ranged from ˜20% in TRV to ˜10% in TAV viral vectors. In addition, we tested for the presence of rolling circle viral replicons in these T1 plants and did not see presence of rolling circles.

The Use of Better CSMs May Increase these Efficiencies and could be a Boon for Researchers Working with Tomato and Other Solanaceae.

In conventional CRISPR experiments that targeted more than one locus, the frequency of finding mutations at both loci was shown to be higher (Cermak et al., 2017). We further analyzed the frequency of mutations at HSVtk and the second locus C upon counter-selection. In our experiments we used the C-locus as the second site because mutations at this locus produce a visible potato leaf phenotype (FIG. 11). Our analysis is based on this visible phenotype. Only mutations in both copies of the C gene produce the potato leaf phenotype. However, it is possible that other normal looking shoots contain one mutated allele, and hence would lead to higher mutation efficiency. We checked the HSVtk resistant shoots for heterozygous and homozygous mutations at the C locus. We also determined that transiently expressing Cas9, in addition to the stably expressed Cas9 in the transgenic stock, increases the efficiency of CRISPR mutagenesis. Current ganciclovir concentrations produce a number of escapes (current concentrations produce ˜60% escapes). However, it is worth pointing out that, getting editing in ˜40% of the regenerated shoots is already quite remarkable! Selected double mutants at HSVtk and C have been taken to the next generation to test for heritability. In addition, we tested for the presence of rolling circle viral replicons and did not see presence of rolling circles in these plants.

Preliminary tests in soybean, pepper, cacao, sunflower, and coffee: Decapitated soybean, and coffee seedlings produce new shoots upon decapitation. Soybean, cacao, pepper, and sunflower were tested for marker GUS expression after infiltration with our pTAV viral replicon vectors and GUS expression was detected in all four species.

We have demonstrated efficient expression of a marker transgene (GUS) when delivered by our methodology (Agrobacterium injection into the stem, transfer by Agrobacterium of a T-DNA carrying a viral replicon into the plant, and expression of a marker gene carried on the replicon). This indicates that expression of the targeting endonuclease in these plants will also be efficient. In each of these cases regeneration of shoots from the decapitation site was also efficient. Non-limiting examples of other crop species with excellent expression and regeneration include the crops pepper (Capsicum annuum) and eggplant (Solanum melongela), and the more diverged common bean (Phaseolus vulgaris).

### The Use of KU80/Ku70 to Increase Frequency of Genome Editing and Gene Targeting by Recombination.

Gene editing is achieved by the induction of double strand breaks, which are then repaired via host-encoded processes. If a break is incorrectly repaired, then a mutation occurs. If repair is error-free, then the target is restored and may be cleaved again provided the editing elements are still present.

Plants, alike other living things, possess a variety of DSB repair pathways, only some of which are mutagenic. Under some circumstances, repair occurs via the copying of information from an intact but homologous sequence elsewhere in the genome. However, such homology-dependent processes are rare in mitotic cells. In S and G2-phase mitotic cells homology dependent repair of breaks occurs by copying of intact sister chromatid sequences-such repair is error-free and therefore does not result in mutagenesis (and therefore restores the target, for possible recutting later). In G1 cells, when no sister chromatid is available, repair occurs via one of several possible nonhomologous end-joining pathways. The canonical pathway, considered to be the most efficient pathway in most eukaryotes, requires the ku heterodimer (ku70+ku80), DNA ligase IV, and XRCC4 proteins. These 4 proteins act together to protect the broken ends from degradation (or sequestration by alternative pathways) and re-ligate the break. Recent evidence has demonstrated (in nematodes—van Schendel, Roerink, Portegijs, van den Heuvel, & Tijsterman, 2015) that this canonical pathway is extremely efficient, fast, and largely error free. In other words, the majority of breaks that might lead to mutation are instead immediately protected by the ku dimer, which is expressed at remarkably high concentrations in the cell. It has recently been demonstrated—in nematode worms—that CRISPR mutagenesis is entirely dependent on what is considered to be a “backup” NHEJ pathway, which requires DNA polymerase theta (aka polQ). Pol theta is a nonprocessive and relatively error-prone polymerase that has the ability to prime DNA synthesis with using only one or two base-paired nucleotides, and has recently been shown to be entirely responsible for T-DNA integration in plants (van Kregten et al., 2016), although data on its role in CRISPR induced mutagenesis has not yet been published. The polQ-dependent pathway may be Ku-independent. For this reason we propose that plants carrying a knockout allele for KU80/Ku70 will experience earlier and more efficient CRISPR mutagenesis, as the break will be processed instead by more error-prone polQ-dependent pathway. We have generated tomato lines, now in the T1 generation, that are homozygous for a KO mutation in PolQ and heterozygous for KU80. We have both Cas9+ and Cas9 null segregants and will soon be able to test the effects of each of these mutations on CRISPR-induced editing.

TABLE 4
Regeneration of plants treated with DAAs through different application methods

| | | | | Transgene- | No treatment |
|---|---|---|---|---|---|
| | Application | DAAs | DAAO Plants | free Plants | DAAO Plants |
| Test | Method | Concentration | Regeneration | Regeneration | Regeneration |
| 1 | Lanolin | 30 mM D-Val, | 13 out of 16 | 8 out of 8 | 11 out of 11 |
| | paste | 40 mM D-Ile | (81%) | (100%) | (100%) |
| | Irrigation | 30 mM D-Val, | 10 out of 15 | 8 out of 8 | |
| | | 40 mM D-Ile | (67%) | (100%) | |
| 2 | Lanolin | 30 mM D-Val, | 7 out of 10 | 6 out of 6 | 16 out of 19 |
| | paste | 40 mM D-Ile | (70%) | (100%) | (84%) |
| | Agarose | 30 mM D-Val, | 12 out of 13 | 5 out of 6 | |
| | gel | 40 mM D-Ile | (92%) | (83%) | |

TABLE 5
Segregation analysis and phenotypes of different DAAO transgenic
lines Plants described in the table are all T₁ plants.

| | Genotype | | Phenotype |
|---|---|---|---|
| DAAO transgenic | Percent DAAO | Percent transgene- | Percent weird |
| line | transgenic | free | plants |
| DAAO line 1 | 91.5% | 8.5% | 47% |
| DAAO line 18 | 90.4% | 9.6% | 43% |
| DAAO line 4 | 66.7% | 33.3% | 4% |

TABLE 6
Toxicity of GAN to HSVtk plants
Transgene-free plants were included as controls.
They were treated with the same concentration
of GAN as the HSVtk plants in each group.

| | | |
|---|---|---|
| | | Regeneration |
| Concentration | Regeneration | of transgene- |
| of GAN | of HSVtk | free plants |
| applied | plants | (control) |

| | | | |
|---|---|---|---|
| 0 | mM | 94% | 100% |
| | | (16 out of 17) | (15 out of 15) |
| 0.1 | mM | 89% | 93% |
| | | (16 out of 18) | (14 out of 15) |
| 1 | mM | 25% | 53% |
| | | (4 out of 16) | (8 out of 15) |
| 4.5 | mM | 0% | 26% |
| | | (0 out of 10) | (6 out of 23) |

Sequences
SEQ ID NO: 29
pDe/Kan-Cas9-DAAO
SEQ ID NO: 30
pDe/Kan-Cas9-HSVtk
SEQ ID NO: 31
pMP6 (pTAV-DAAO.ENT.tRNA)
SEQ ID NO: 32
pMP4 (pTRV2e-DAAO.ENT.tRNA)
SEQ ID NO: 33
pMR316_pTAVbinary_GUSPlus
SEQ ID NO: 34
pTRV2e-ER_tagRFP
SEQ ID NO: 35
ENTIRE-ATG to STOP
SEQ ID NO: 36
Potato Leaf (C)-ATG to STOP

### REFERENCES

Adli, M. (2018). The CRISPR tool kit for genome editing and beyond. Nature Communications, 9(1), 1911. http://doi.org/10.1038/s41467-018-04252-2
Ali, Z., Abul-faraj, A., Li, L., Ghosh, N., Piatek, M., Mahjoub, A., et al. (2015). Efficient Virus-Mediated Genome Editing in Plants Using the CRISPR/Cas9 System. Molecular Plant, 8(8), 1288-1291. http://doi.org/10.1016/j.molp.2015.02.011
Ali, Zahir, et al. “Activity and specificity of TRV-mediated gene editing in plants.” Plant signaling & behavior 10.10 (2015): e1044191.
Alonso, Jorge, et al. “D-Amino-acid oxidase gene from Rhodotorula gracilis (Rhodosporidium toruloides) ATCC 26217.” Microbiology 144.4 (1998): 1095-1101.
Amutha, S., Kathiravan, K., Singer, S., Jashi, L., Shomer, I., Steinitz, B., & Gaba, V. (2009). Adventitious shoot formation in decapitated dicotyledonous seedlings starts with regeneration of abnormal leaves from cells not located in a shoot apical meristem. In Vitro Cellular & Developmental Biology—Plant, 45(6), 758-768. http://doi.org/10.1007/s11627-009-9232-8
Baltes, N. J., Gil-Humanes, J., Cermak, T., Atkins, P. A., & Voytas, D. F. (2014). DNA replicons for plant genome engineering. The Plant Cell Online, 26(1), 151-163. http://doi.org/10.1105/tpc.113.119792
Bedell, V. M., Wang, Y., Campbell, J. M., Poshusta, T. L., Starker, C. G., Krug, R. G. 2., et al. (2012). In vivo genome editing using a high-efficiency TALEN system. Nature, 491(7422), 114-118. http://doi.org/10.1038/nature11537
Belhaj, K., Chaparro-Garcia, A., Kamoun, S., & Nekrasov, V. (2013). Plant genome editing made easy: targeted mutagenesis in model and crop plants using the CRISPR/Cas system. Plant Methods, 9(1), 39. http://doi.org/10.1186/1746-4811-9-39
Bortesi, L., & Fischer, R. (2015). The CRISPR/Cas9 system for plant genome editing and beyond. Biotechnology Advances, 33(1), 41-52. http://doi.org/10.1016/j.biotechadv.2014.12.006
Brooks, C., Nekrasov, V., Lippman, Z. B., & Van Eck, J. (2014). Efficient Gene Editing in Tomato in the First Generation Using the Clustered Regularly Interspaced Short Palindromic Repeats/CRISPR-Associated9 System. Plant Physiology, 166(3), 1292-1297. http://doi.org/10.1104/pp. 114.247577
Campbell, B. W., Hofstad, A. N., Sreekanta, S., Fu, F., Kono, T. J. Y., O'Rourke, J. A., et al. (2016). Fast neutron-induced structural rearrangements at a soybean NAP1 locus result in gnarled trichomes. TAG Theoretical and Applied Genetics Theoretische Und Angewandte Genetik, 129(9), 1725-1738. http://doi.org/10.1007/s00122-016-2735-x
Cermak, T., Baltes, N. J., Čegan, R., Zhang, Y., & Voytas, D. F. (2015). High-frequency, precise modification of the tomato genome. Genome Biology, 16(1), 232. http://doi.org/10.1186/s13059-015-0796-9
Cermak, T., Curtin, S. J., Gil-Humanes, J., Čegan, R., Kono, T. J. Y., Konečná, E., et al. (2017). A Multipurpose Toolkit to Enable Advanced Genome Engineering in Plants. The Plant Cell Online, 29(6), 1196-1217. http://doi.org/10.1105/tpc.16.00922
Chaudhary, J., Patil, G. B., Sonah, H., Deshmukh, R. K., Vuong, T. D., Valliyodan, B., & Nguyen, H. T. (2015). Expanding Omics Resources for Improvement of Soybean Seed Composition Traits. Frontiers in Plant Science, 6(31), 1021. http://doi.org/10.3389/fpls.2015.01021
Chen, Ju-Jiun, et al. “A gene fusion at a homeobox locus: alterations in leaf shape and implications for morphological evolution.” The Plant Cell Online 9.8 (1997): 1289-1304.
Cordell, D., Drangert, J.-O., & White, S. (2009). The story of phosphorus: Global food security and food for thought. Global Environmental Change, 19(2), 292-305. http://doi.org/10.1016/j.gloenvcha.2008.10.009
Czakó, M., & Márton, L. (1994). The herpes simplex virus thymidine kinase gene as a conditional negative-selection marker gene in Arabidopsis thaliana. Plant Physiol, 104(3), 1067-1071.
Czakó, M., Marathe, R. P., Xiang, C., Guerra, D. J., Bishop, G. J., Jones, J. D., & Márton, L. (1995). Variable expression of the herpes simplex virus thymidine kinase gene in Nicotiana tabacum affects negative selection. TAG Theoretical and Applied Genetics Theoretische Und Angewandte Genetik, 91(8), 1242-1247. http://doi.org/10.1007/BF00220935
Dahan-Meir, T., Filler-Hayut, S., Melamed-Bessudo, C., Bocobza, S., Czosnek, H., Aharoni, A., & Levy, A. A. (2018). Efficient in plantagene targeting in tomato using geminiviral replicons and the CRISPR/Cas9 system. The Plant Journal, 8, 1288. http://doi.org/10.1111/tpj.13932
Erikson, O., Hertzberg, M., & Näsholm, T. (2004). A conditional marker gene allowing both positive and negative selection in plants. Nature Biotechnology, 22(4), 455-458.
Fedoroff, N. V., Fedoroff, N. V., Battisti, D. S., Battisti, D. S., Beachy, R. N., Beachy, R. N., et al. (2010). Radically rethinking agriculture for the 21st century. Science (New York, N.Y.), 327(5967), 833-834. http://doi.org/10.1126/science.1186834
Food and Agriculture Organization of the United Nations. (2013. How to Feed the World in 2050. Retrieved Jul. 31, 2013, from http://www.fao.org/fileadmin/templates/wsfs/docs/expert_paper/How_to_Feed_the_Worl d_in_2050.pdf
Gilbertson, R. L., Hidayat, S. H., Paplomatas, E. J., Rojas, M. R., Hou, Y. M., & Maxwell, D. P. (1993). Pseudorecombination between infectious cloned DNA components of tomato mottle and bean dwarf mosaic geminiviruses. Journal of General Virology, 74(1), 23-31. http://doi.org/10.1099/0022-1317-74-1-23
Hanley-Bowdoin, Linda, et al. “Geminiviruses: masters at redirecting and reprogramming plant processes.” Nature Reviews. Microbiology 11.11 (2013): 777.
Horiguchi, G., Kim, G.-T., & Tsukaya, H. (2005). The transcription factor AtGRF5 and the transcription coactivator AN3 regulate cell proliferation in leaf primordia of Arabidopsis thaliana. The Plant Journal: for Cell and Molecular Biology, 43(1), 68-78. http://doi.org/10.1111/j.1365-313X.2005.02429.x
Hou, Y.-M., Paplomatas, E. J., & Gilbertson, R. L. (1998). Host Adaptation and Replication Properties of Two Bipartite Geminiviruses and Their Pseudorecombinants. Molecular Plant-Microbe Interactions, 11(3), 208-217. http://doi.org/10.1094/MPMI.1998.11.3.208
Jinek, M., Chylinski, K., Fonfara, I., Hauer, M., Doudna, J. A., & Charpentier, E. (2012). A programmable dual-RNA-guided DNA endonuclease in adaptive bacterial immunity. Science (New York, N.Y.), 337(6096), 816-821. http://doi.org/10.1126/science.1225829
Johkan, M., Mori, G., Imahori, Y., Mitsukuri, K., Yamasaki, S., Mishiba, K., et al. (2008). Shading the Cut Stems of Tomato Plants Promotes in vivo Shoot Regeneration via Control of the Phenolic Metabolism. Environment Control in Biology, 46(3), 203-209. http://doi.org/10.2525/ecb.46.203
Knapp, A. K., Beier, C., Briske, D. D., Classen, A. T., Luo, Y., Reichstein, M., et al. (2008). Consequences of More Extreme Precipitation Regimes for Terrestrial Ecosystems. BioScience, 58(9), 811. http://doi.org/10.1641/B580908
Koenig, D., Bayer, E., Kang, J., Kuhlemeier, C., & Sinha, N. (2009). Auxin patterns Solanum lycopersicum leaf morphogenesis. Development (Cambridge, England), 136(17), 2997-3006. http://doi.org/10.1242/dev.033811
Liao, S., Tammaro, M., & Yan, H. (2015). Enriching CRISPR-Cas9 targeted cells by co-targeting the HPRT gene. Nucleic Acids Research, 43(20), e134. http://doi.org/10.1093/nar/gkv675
Lin, J.-Y., Le, B. H., Chen, M., Henry, K. F., Hur, J., Hsieh, T.-F., et al. (2017). Similarity between soybean and Arabidopsis seed methylomes and loss of non-CG methylation does not affect seed development. Proc Natl Acad Sci USA, 114(45), E9730—E9739. http://doi.org/10.1073/pnas.1716758114
Liu, Yule, Michael Schiff, and S. P. Dinesh-Kumar. “Virus-induced gene silencing in tomato.” The Plant Journal 31.6 (2002): 777-786.
Liu, Yule, et al. “Tobacco Rar1, EDS1 and NPR1/NIM1 like genes are required for N-mediated resistance to tobacco mosaic virus.” The Plant Journal 30.4 (2002): 415-429.
Ma, X., Zhang, Q., Zhu, Q., Liu, W., Chen, Y., Qiu, R., et al. (2015). A Robust CRISPR/Cas9 System for Convenient, High-Efficiency Multiplex Genome Editing in Monocot and Dicot Plants. Molecular Plant, 8(8), 1274-1284. http://doi.org/10.1016/j.molp.2015.04.007
Maher, K. A., Bajic, M., Kajala, K., Reynoso, M., Pauluzzi, G., West, D. A., et al. (2018). Profiling of Accessible Chromatin Regions across Multiple Plant Species and Cell Types Reveals Common Gene Regulatory Principles and New Control Modules. The Plant Cell . . . , 30(1), 15-36. http://doi.org/10.1105/tpc.17.00581
Moyle, L. C. (2008). Ecological and evolutionary genomics in the wild tomatoes (Solanum sect. Lycopersicon). Evolution, 62(12), 2995-3013. http://doi.org/10.1111/j.1558-5646.2008.00487.x
Nakayama, H., Sakamoto, T., Okegawa, Y., Kaminoyama, K., Fujie, M., Ichihashi, Y., et al. (2018). Comparative transcriptomics with self-organizing map reveals cryptic photosynthetic differences between two accessions of North American Lake cress. Scientific Reports, 8(1), 103. http://doi.org/10.1038/s41598-018-21646-w
Nakayama, H., Sinha, N. R., & Kimura, S. (2017). How Do Plants and Phytohormones Accomplish Heterophylly, Leaf Phenotypic Plasticity, in Response to Environmental Cues. Frontiers in Plant Science, 8, 20. http://doi.org/10.3389/fpls.2017.01717
Osakabe, Yuriko, and Keishi Osakabe. “Genome editing with engineered nucleases in plants.” Plant and Cell Physiology 56.3 (2014): 389-400.
Pelletier, J. M., Kwong, R. W., Park, S., Le, B. H., Baden, R., Cagliari, A., et al. (2017). LEC1 sequentially regulates the transcription of genes involved in diverse developmental processes during seed development. Proc Natl Acad Sci USA, 114(32), E6710—E6719. http://doi.org/10.1073/pnas.1707957114
Raitskin, O., & Patron, N. J. (2016). Multi-gene engineering in plants with RNA-guided Cas9 nuclease. Current Opinion in Biotechnology, 37, 69-75. http://doi.org/10.1016/j.copbio.2015.11.008
Reynoso, M. A., Pauluzzi, G. C., Kajala, K., Cabanlit, S., Velasco, J., Bazin, J., et al. (2018). Nuclear Transcriptomes at High Resolution Using Retooled INTACT. Plant Physiology, 176(1), 270-281. http://doi.org/10.1104/pp. 17.00688
Ron, M., Kajala, K., Pauluzzi, G., Wang, D., Reynoso, M. A., Zumstein, K., et al. (2014). Hairy Root Transformation Using Agrobacterium rhizogenes as a Tool for Exploring Cell Type-Specific Gene Expression and Function Using Tomato as a Model. Plant . . . .
Sim, S.-C., Robbins, M. D., Van Deynze, A., Michel, A. P., & Francis, D. M. (2011). Population structure and genetic differentiation associated with breeding history and selection in tomato (Solanum lycopersicum L.). Heredity, 106(6), 927-935. http://doi.org/10.1038/hdy.2010.139
Townsley, B. T., Covington, M. F., & Ichihashi, Y. (2015). BrAD-seq: Breath Adapter Directional sequencing: a streamlined, ultra-simple and fast library preparation protocol for strand specific mRNA library . . . . Frontiers in Plant . . . .
Uchida, N., Townsley, B., Chung, K.-H., & Sinha, N. (2007). Regulation of SHOOT MERISTEMLESS genes via an upstream-conserved noncoding sequence coordinates leaf development. Proceedings of the National Academy of Sciences of the United States of America, 104(40), 15953-15958. http://doi.org/10.1073/pnas.0707577104
van Kregten, M., de Pater, S., Romeijn, R., van Schendel, R., Hooykaas, P. J. J., & Tijsterman, M. (2016). T-DNA integration in plants results from polymerase-θ-mediated DNA repair. Nature Plants, 2(11), 16164. http://doi.org/10.1038/nplants.2016.164
van Schendel, R., Roerink, S. F., Portegijs, V., van den Heuvel, S., & Tijsterman, M. (2015). Polymerase Θ is a key driver of genome evolution and of CRISPR/Cas9-mediated mutagenesis. Nature Communications, 6(1), 7394. http://doi.org/10.1038/ncomms8394
Vassilakos, N., et al. “Tobravirus 2b protein acts in trans to facilitate transmission by nematodes.” Virology 279.2 (2001): 478-487.
Wang, Hua, et al. “The tomato Aux/IAA transcription factor IAA9 is involved in fruit development and leaf morphogenesis.” The Plant Cell Online 17.10 (2005): 2676-2692.
Xie, K., Minkenberg, B., & Yang, Y. (2015). Boosting CRISPR/Cas9 multiplex editing capability with the endogenous tRNA-processing system. Proceedings of the National Academy of Sciences of the United States of America, 112(11), 3570-3575. http://doi.org/10.1073/pnas.1420294112
Xing, Hui-Li, et al. “A CRISPR/Cas9 toolkit for multiplex genome editing in plants.” BMC plant biology 14.1 (2014): 327.
Zhang, Junhong, et al. “A single-base deletion mutation in SlIAA9 gene causes tomato (Solanum lycopersicum) entire mutant.” Journal of plant research 120.6 (2007): 671-678.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A method of generating a plant comprising a mutation in a gene of interest, the method comprising,
providing a plant expressing a guided nuclease targeted to a gene of interest in the plant;
generating a wound at a location on the plant at which the guided nuclease is expressed;
allowing shoots to form from callus at the wound; and
selecting at least one shoot from the wound comprising a guided nuclease-induced mutation in the gene of interest.

2. The method of claim 1, wherein the guided nuclease is a sgRNA-guided nuclease and the plant expresses one or more sgRNA that guides the nuclease to the gene of interest.

3. The method of claim 2, wherein the guided nuclease and the sgRNA are expressed transiently.

4. The method of claim 3, wherein RNA encoding the guided nuclease and the sgRNA are expressed from the same transient vector.

5. (canceled)

6. The method of claim 4, wherein the transient vector is a viral vector.

7. The method of claim 6, wherein the viral vector is a tobacco Rattle Virus (TRV) vector or a Potato Virus X (PVX) vector.

8. The method of claim 3, wherein the providing comprises delivering the guided nuclease and the sgRNA to the plant.

9. The method of claim 8, wherein the guided nuclease and the sgRNA are part of a ribonucloeprotein complex.

10. The method of claim 1, wherein the guided nuclease is expressed from an expression cassette integrated in the genome of the plant.

11. The method of claim 10, wherein the guided nuclease is a sgRNA-guided nuclease and the plant transiently expresses one or more sgRNA that guides the nuclease to the gene of interest.

12. The method of claim 1, wherein the plant further expresses a template nucleic acid molecule that acts as a template for homology-directed recombination (HDR) at the gene of interest after the guided nuclease cleaves the gene of interest.

13. The method of claim 2, further comprising before the generating, expressing a counter-selectable marker in the plant, wherein the counter-selectable marker is shoot meristem-specific,
expressing at least one additional sgRNA at said location, wherein the at least one additional sgRNA targets a gene encoding the counter-selectable marker such that the RNA-guided nuclease inactivates the counter-selectable marker; and
before the selecting, applying counter selection to the plant such that shoots generated at the wound that do not contain the at least one additional sgRNA have inhibited growth compared to shoots that contain the at least one addition sgRNA.

14. The method of claim 13, wherein the counter-selectable marker is a protein that generates a toxic product to plant cell in which the counter-selectable marker is expressed when provided with a substrate.

15. The method of claim 14, wherein the counter-selectable marker is D-amino acid oxidase and the substrate is a D-amino acid.

16. The method of claim 14, wherein the counter-selectable marker is Herpes Simplex Virus-1 Thymidine Kinase (HSVtk) and the substrate is ganciclovir.

17. The method of claim 1, wherein the plant is a monocot.

18. The method of claim 1, wherein the plant is a dicot.

19. (canceled)

20. The method of claim 1, wherein the plant is knocked-out for, has reduced or inhibited expression of, has reduced or inhibited activity of, or contains an inactivating mutation in at least one of more of ku70, ku80, DNA ligase IV, polQ, or XRCC4 protein.

21. The method of claim 1, further comprising regenerating a plant from a shoot selected as comprising the guided nuclease-induced mutation in the gene of interest.

22. A plant comprising callus at a wound site generated by removal of a shoot, the wound comprising a guided nuclease targeting a gene of interest, wherein the callus comprises one or more shoot comprising a mutated copy of the gene of interest, wherein the mutated copy was generated by cleavage of the gene of interest by the guided nuclease.

23-39. (canceled)
